(19) United States
(12) Patent Application Publication
(10) Pub. No.: US 2003/0190624 A1
(43) Pub. Date: Oct. 9, 2003

(54) Genes expressed with high specificity in kidney

(76) Inventors: Chao Zhang, Moraga, CA (US); Junming Yang, San Jose, CA (US); Michael G. Walker, Sunnyvale, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/113,644

(22) Filed: Mar. 26, 2002

(51) Int. Cl. C12Q 1/68; G01N 33/53; C12P 21/04; C12N 9/00; C12P 21/02; C12N 5/06; C07K 14/47; C07K 16/40

(52) U.S. Cl. 435/6; 435/7.1; 435/69.1; 435/320.1; 435/325; 435/183; 530/350; 530/388.1; 435/70.21

(57) ABSTRACT

The invention provides polynucleotides that are specifically expressed in kidney function or kidney disorders. The invention also provides compositions, probes, expression vectors, host cells, proteins encoded by the polynucleotides and agonist, antagonists and antibodies which specifically bind the proteins. The invention also provides methods for assessing kidney function and for the diagnosis, prognosis, treatment and evaluation of therapies for kidney disorders.

### FIELD OF THE INVENTION

The invention relates to isolated polynucleotides and proteins that are highly, specifically expressed in kidney and useful for assessing kidney function or in diagnosing, staging, treating and evaluating therapies for kidney disorders.

### BACKGROUND OF THE INVENTION

The kidney is the organ primarily responsible for removing soluble waste products from the blood. The cells of the kidney express a variety of genes that regulate or participate in the elimination of such substances as drugs, minerals, hormones, and nutrients from the blood and in the regulation of blood pressure, blood volume, and electrolyte concentrations.

Urine formation is a balance between glomerular filtration and tubular re-absorption/secretion. The kidney has developed high-capacity transport systems to prevent loss of nutrients as well as electrolytes and to facilitate tubular secretion of a wide range of organic ions. The dysfunction of a transport system often leads to kidney dysfunction or failure.

Over the past few years, a steadily increasing number of kidney-specific genes have been identified. The characterization of these genes sheds light on the important functions of kidney and reveals long-sought links between genes and diseases. For example, the inherited renal tubular disorders associated with hypokalemic alkalosis (Bartter Syndromes) have been attributed to mutations in several kidney-specific transporter genes (Simon and Lifton (1998) Curr Opin Cell Biol 10:450-454). The Gitelman variant of Bartter syndrome (MIM 263800) is uniformly caused by mutations in the gene for the thiazide-sensitive Na—Cl cotransporter (NCC), while antenatal variant of Bartter syndrome is caused by mutations in the gene for either the furosemide-sensitive N—K-2Cl cotransporter NKCC2 (MIM 600839) or the inwardly-rectifying potassium channel ROMK1 (MIM 600359). One of the recently identified genes, NPHS1, encodes a transmembrane protein that is exclusively localized at the slit diaphragm of the interdigitated podocyte foot processes (Holthofer et al. (1999) Am J Pathol 155:1681-1687). NPHS1 is mutated in congenital nephrotic syndrome of the Finnish type (CNF, MIM 256300), the most severe genetic disorder with filtration barrier defects (Kestila et al. (1998) Mol Cell 1:575-582).

Kidney transport systems also have a direct impact on drug metabolism. Disposition of drugs is the consequence of interaction with diverse secretory and absorptive transporters in renal tubules (Inui et al. (2000) Kidney Int 58:944-058). The identification and functional characterization of drug transporters provides valuable information regarding the cellular network involved drug catabolism. The limited availability of human kidney tissue (for ethical and technical reasons) increases the difficulty in evaluating potential therapeutic compounds in vitro. These efforts are also hindered by the difficulty of extrapolating experimental results from animal models or immortalized cell lines to the effects in vivo in humans. The ability to grow kidney tissue from stem cells and maintain them in culture would greatly increase the ability to develop and test drugs. Genes that can serve as markers for the differentiation of stem cells into kidney tissue, or that may induce or maintain differentiation, are useful experimentally and, perhaps, therapeutically.

Given the current state of knowledge, pharmaceutical and medical needs, the identification of previously-uncharacterized genes that are expressed with high specificity in kidney satisfies a need in the art by providing a combination of polynucleotides and compositions comprising polynucleotides, their encoded proteins, and antibodies that specifically bind the proteins, each of which may be used to induce, maintain or monitor the differentiation of kidney cells and tissues from stem cells, evaluate kidney function, and in the diagnosis, prognosis, treatment and evaluation of therapies for kidney disorders.

### SUMMARY OF THE INVENTION

The invention provides a combination comprising a plurality of polynucleotides wherein the plurality of polynucleotides have the nucleic acid sequences of SEQ ID NOs: 3-18 that are specifically expressed in kidney disorders or the complements of SEQ ID NOs: 3-18. The invention also provides an isolated polynucleotide having a nucleic acid sequence selected from SEQ ID NOs: 3-18 and the complements thereof. In different aspects, each polynucleotide is used as a diagnostic, as a probe, in an expression vector, and in assessing kidney function or the prognosis and treatment of kidney disorders.

The invention provides a method of using a combination or an isolated polynucleotide to screen a plurality of molecules to identify at least one ligand which specifically binds a polynucleotide, the method comprising contacting the combination or the polynucleotide with molecules under conditions to allow specific binding; and detecting specific binding, thereby identifying a ligand which specifically binds the polynucleotide. In one embodiment, the molecules are selected from DNA molecules, RNA molecules, peptide nucleic acids, peptides, and proteins. The invention further provides a method for using a combination or an isolated polynucleotide to detect expression in a sample containing nucleic acids, the method comprising hybridizing the combination or polynucleotide to the nucleic acids under conditions for formation of one or more hybridization complexes; and detecting hybridization complex formation, wherein complex formation indicates expression in the sample. In one embodiment, the polynucleotides are attached to a substrate. In another embodiment, the sample is from kidney. In yet another embodiment, the nucleic acids are amplified prior to hybridization. In still another embodiment, complex formation is compared to standards and is diagnostic of kidney function or kidney disorders including, but not limited to, Addison's disease, Bartter syndrome, cancer including renal cell carcinoma, clear cell carcinoma, Wilms' tumor, hypernephroma, and inflammatory complications of cancer, Gitelman syndrome, hypertension, hypotension, hypocalciuria, glomerulonephritis, congenital nephrotic syndrome, interstitial nephritis, nephrolithiasis, polycystic kidney disease, renal failure, renal tubule acidosis, and complications of kidney transplant.

The invention provides a vector containing the polynucleotide, a host cell containing a vector and a method for using a host cell to produce a protein or peptide encoded by the polynucleotide comprising culturing the host cell under conditions for expression of the protein and recovering the protein from cell culture. The invention also provides purified proteins, SEQ ID NOs: 1 and 2, encoded by polynucleotides of the invention. The invention further provides a method for using the protein or peptide to screen a plurality of molecules to identify at least one ligand which specifically binds the protein. In one embodiment, the molecules to be screened are selected from agonists, antagonists, antibodies, DNA molecules, RNA molecules, peptides, peptide nucleic acids, and proteins.

The invention provides a method of using a protein or peptide to identify an antibody which specifically binds the protein, the method comprising contacting a plurality of antibodies with the protein under conditions for formation of an antibody:protein complex, and dissociating the antibody from the antibody:protein complex, thereby obtaining antibody which specifically binds the protein. In one aspect, the plurality of antibodies are selected from polyclonal antibodies, monoclonal antibodies, chimeric antibodies, recombinant antibodies, humanized antibodies, single chain antibodies, Fab fragments, F(ab′)₂ fragments, Fv fragments and antibody-peptide fusion proteins. The invention also provides methods for preparing and purifying antibodies. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides purified antibodies which specifically bind a protein. The invention also provides a method for using an antibody to detect expression of a protein in a sample, the method comprising combining the antibody with a sample under conditions for formation of antibody: protein complexes; and detecting complex formation, wherein complex formation indicates expression of the protein in the sample. In one aspect, the amount of complex formation when compared to standards is diagnostic of kidney function or kidney disorders. In another aspect, the antibody is part of an array. The invention further provides a method for immunopurification of a protein comprising attaching an antibody to a substrate, exposing the antibody to a sample containing protein under conditions to allow antibody: protein complexes to form, dissociating the protein from the complex, and collecting purified protein.

The invention provides a composition comprising a polynucleotide, a protein, or an antibody that specifically binds a protein or peptide for use in detecting or treating kidney disorders.

### BRIEF DESCRIPTION OF THE SEQUENCE LISTING

The Sequence Listing provides SEQ ID NOs: 3-18, exemplary polynucleotides of the invention. Each sequence is identified by a sequence identification number (SEQ ID NO) and by the Incyte number with which the sequence was first identified.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two polynucleotides, proteins, or antibodies on a substrate. At least one of the polynucleotides, proteins, or antibodies represents a control or standard, and the other polynucleotide, protein, or antibody is of diagnostic or therapeutic interest. The arrangement of at least two and up to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable.

The “complement” of a polynucleotide of the Sequence Listing refers to a nucleic acid molecule which is completely complementary over its full length and which will hybridize to a nucleic acid or an mRNA under conditions of high stringency.

“Differential expression” refers to an increased or upregulated or a decreased or downregulated expression as detected by presence, absence or at least about a two-fold change in the amount of protein or mRNA in a sample.

“Isolated or purified” refers to a polynucleotide, protein or antibody that is removed from its natural environment and that is separated from other components with which it is naturally present.

A “composition” refers to the polynucleotide and a labeling moiety; a purified protein and a pharmaceutical carrier or a heterologous, labeling or purification moiety; an antibody and a labeling moiety or pharmaceutical agent; and the like.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses labeling moieties and/or antibodies to detect expression in a sample. The nucleic acids, proteins, or antibodies may be used in solution or attached to a substrate, and their detection is based on methods well known in the art.

A “hybridization complex” is formed between a polynucleotide and a nucleic acid of a sample when the purines of one molecule hydrogen bond with the pyrimidines of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions.

“Identity”, as applied to nucleic and amino acid sequences, refers to the quantification (usually percentage) of nucleotide or residue matches between at least two sequences aligned using a standardized algorithm such as Smith-Waterman (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402. BLAST2 may be used in a standardized and reproducible way to insert gaps in one of the sequences in order to optimize alignment and to achieve a more meaningful comparison between them. “Similarity” uses the same algorithms but takes conservative substitution of nucleotides and residues into account. In proteins, similarity exceeds identity in that substitution of a valine for a leucine or isoleucine, is counted in calculating the reported percentage. Substitutions which are considered to be conservative are well known in the art.

“Isolated or “purified” refers to any molecule or compound that is separated from its natural environment and is from about 60% free to about 90% free from other components with which it is naturally associated.

“Kidney disorders” include conditions, diseases and syndromes which affect the kidneys. They include Addison's disease, Bartter syndrome, cancer including renal cell carcinoma, clear cell carcinoma, Wilms' tumor, hypernephroma, and inflammatory complications of cancer, Gitelman syndrome, hypertension, hypotension, hypocalciuria, glomerulonephritis, juvenile nephronophthisis, congenital nephrotic syndrome, interstitial nephritis, nephrolithiasis, polycystic kidney disease, renal failure, renal tubule acidosis, and complications of kidney transplant.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Markers for kidney” refers to polynucleotides and proteins which are specifically expressed in the development, differentiation, and function of kidney cells and tissues and in the diagnosis, prognosis, treatment or evaluation of therapies for kidney diseases.

“Polynucleotide” refers to a chain of nucleotides, a nucleic acid, or an isolated cDNA. It may be of recombinant or synthetic origin, double-stranded or single-stranded, and combined with vitamins, minerals, carbohydrates, lipids, proteins, or other nucleic acids to perform a particular activity or form a useful composition.

The phrase “polynucleotide encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

“Probe” refers to a polynucleotide that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis (Washington University, St. Louis Mo.) or an antigenic determinant of the protein identified using Kyte-Doolittle algorithms of the PROTEAN program (DNASTAR, Madison Wis.).

“Sample” is used in its broadest sense as containing nucleic acids, proteins, and antibodies. A sample may comprise a bodily fluid such as blood, lymph, spinal fluid, sputum, or urine; the soluble fraction of a cell preparation, or an aliquot of media in which cells were grown; a chromosome, an organelle, or membrane isolated or extracted from a cell; genomic DNA, cDNA, nucleic acids, polynucleotides, or RNA, in solution or bound to a substrate; a cell; a tissue; a tissue print; buccal cells, skin, hair follicle; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which polynucleotides, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is an expression profile of transcriptional activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encodes it. Splice variants may be determined by BLAST score, wherein the score is at least 100, and most preferably at least 400. Allelic variants have a high percent identity to the polynucleotides and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The present invention identifies a plurality of polynucleotides, and their encoded proteins or peptides, that are significantly co-expressed with genes known to function in the kidney. These previously uncharacterized biomolecules are useful: 1) as markers for the differentiation of embryonic or adult stem cells into kidney cells and tissues; 2) in the testing, identification, or evaluation of compounds that induce, or prevent, differentiation of stem cells into kidney cells and tissues; 3) as surrogate diagnostic markers for known genes involved in kidney disorders; 4) as high-priority candidates in the search for mutations that cause kidney disorders or as indicators of kidney-cell damage induced by drugs or environmental toxins; and as potential therapeutics for kidney disorders. Four of the polynucleotides have homologs in the public domain databases and eleven are novel. Two proteins encoded by polynucleotides of the invention are also described and characterized in EXAMPLES V and XI.

The method disclosed below provides for the identification of polynucleotides that are expressed in a plurality of libraries. The polynucleotides originate from human cDNA libraries derived from a variety of sources. These polynucleotides can also be selected from a variety of sequence types including, but not limited to, expressed sequence tags (ESTs), assembled polynucleotides, full length coding regions, promoters, introns, enhancers, 5′ untranslated regions, and 3′ untranslated regions.

The cDNA libraries used in the analysis can be obtained from any human tissue including, but not limited to, adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleura, prostate, salivary glands, seminal vesicles, skeleton, spleen, stomach, testis, thymus, tongue, ureter, and uterus.

The polynucleotides are highly specific to and differentially expressed in cells and tissues of kidney. The tissue distribution of 40,285 gene bins in 1222 libraries in the LIFESEQ GOLD database (release October 2000; Incyte Genomics, Palo Alto Calif.) were analyzed. The 40,285 gene bins represent cDNAs that were detected in at least 5 of 1292 libraries. The 1222 libraries include all surgical samples, biopsies, and cell line cDNA libraries and are the subset of 1292 libraries that had unique tissue types. cDNA libraries which were constructed using tissues described as either mixed or pooled were not used in this analysis.

In a preferred embodiment, the polynucleotides are assembled from related sequences, such as sequence fragments derived from a single transcript. Assembly of the polynucleotide can be performed using sequences of various types including, but not limited to, ESTs, extension of the EST, shotgun sequences from a cloned insert, or full length cDNAs. In a most preferred embodiment, the polynucleotides are derived from human sequences that have been assembled using the algorithm disclosed in U.S. Pat. No. 9,276,534, filed Mar. 25, 1999, incorporated herein by reference.

Experimentally, an expression profile which shows the specific and differential expression of the polynucleotides or proteins can be evaluated by methods including, but not limited to, differential display by spatial immobilization or by gel electrophoresis, genome mismatch scanning, representational discriminant analysis, nucleotide, protein, or antibody array analysis, quantitative PCR, and transcript imaging. Any of these methods can be used alone or in combination, and at least two methods are demonstrated for some of the claimed polynucleotides.

The Method

The method for identifying polynucleotides that exhibit a specific and statistically significant expression pattern in kidney, and specifically in kidney function and disorders, is presented below. First, the presence or absence of a polynucleotide in a cDNA library is defined: a polynucleotide is present when at least one cDNA fragment corresponding to that polynucleotide is detected in a cDNA sample taken from the library, and a polynucleotide is absent when no corresponding cDNA fragment is detected in the sample. This method was applied to the data in the LIFESEQ GOLD database (Incyte Genomics).

To determine whether a polynucleotide (G) is kidney specific, two statistical tests are applied. In the first test, the significance of gene expression is evaluated using a probability method to measure a due-to-chance probability of expression. Two dichotomous variables are used to classify the 1222 cDNA libraries, X which determines whether G is present (P) or absent (A), and Y which determines whether the cDNA library is from kidney (K) or not (θ). Occurrence data in the various categories is summarized in the following 2×2 contingency table.

| | Kidney | Non-kidney |
|---|---|---|
| G present | PK | Pθ |
| G absent | AK | Aθ |

If polynucleotide G is kidney specific, a positive association between the two variables X and Y is expected; that is, a significant number of libraries should fall into the PK and Aθ categories. To evaluate the significance in statistical terms, the following question is asked: if the null hypothesis were true—that is, the presence of polynucleotide G were completely independent of whether the tissue is kidney or not—how likely is it that the result occurred by chance. This is provided by applying the Fisher Exact Test and for examining the p-value (Agresti (1990) Categorical Data Analysis, John Wiley & Sons, New York N.Y.; Rice (1988) Mathematical Statistics and Data Analysis, Duxbury Press, Pacific Grove Calif.). The smaller the P value, the less likely that the association between X and Y is due-to-chance.

To illustrate, if a polynucleotide (Incyte 334445; g639841 which is renal Na+-dependent phosphate cotransporter) was detected in eight of the 1222 cDNA libraries and seven of those were from kidney, the corresponding contingency table would be:

| | Kidney | Non-kidney |
|---|---|---|
| G present | 7 | 1 |
| G absent | 38 | 1174 |

and the Fisher Exact Test, would provide a p-value of 4.4e−10, which indicates that the polynucleotide is kidney-specific.

In the second test, the EST counts of polynucleotide G from all libraries that were taken from the same tissue are combined, and the sum is used as a measure of the expression level in that tissue. In particular, the combined EST count of G in kidney libraries (N_{GK}) is compared to the total number of ESTs for all polynucleotides which occur in breast libraries (N_{K}) to derive an estimate of the relative abundance of G transcripts in kidney. Similarly, the combined EST count of G in non-kidney libraries (N_{GK}) is compared with the total number of ESTs in non-kidney libraries (N_{θ}). These values are used to define a likelihood score

L=log2(N_{GK}/N_{K})/(N_{Gθ}/N_{θ}),

which reflects how many times more likely it is for the transcript of polynucleotide G to be found in kidney versus non-kidney tissue. For the polynucleotide shown in the contingency table above, the respective counts are N_{GK}=13, N_{K}=159485, N_{Gθ}=1, and N_{θ}=3506047, which give rise to L=log2(260)=8.16. Because the likelihood score is susceptible to the counting errors that exist in some libraries, the likelihood score is only used as a secondary measure.

In other words, polynucleotides with a significant p-value of P<1e−6, are only considered to be kidney-specific if L>5.5. Experimentally, this two-step filtering process selected most polynucleotides known to function in kidney without including any false positives. Note, however, that the definition of L is flawed when N_{GK}=0 or N_{Gθ}=0 (i.e., L>5.5 is considered only when N_{Gθ} and N_{GK}≠0).

Using this method, those polynucleotides that exhibit significant association with kidney have been identified. Their expression patterns were compared with those of known kidney genes and diagnostic markers using the Guilt-by-Association (GBA) analysis for co-expression patterns described by Walker et al. (1999; Genome Res 9:1198-203; incorporated herein by reference). The known diagnostic markers highly significantly co-express with the polynucleotides of the invention. Therefore, the polynucleotides of the invention are useful to assess kidney function and as surrogate markers for the diagnosis, prognosis, treatment and evaluation of therapies for kidney disorders. Further, the polynucleotides, a protein or peptide encoded by the polynucleotides, or an antibody that specifically binds any of the encoded proteins or peptides can be used as diagnostic markers, potential therapeutics, or targets for the identification, development, or monitoring of therapeutics.

In one embodiment, the invention encompasses a combination comprising a plurality of polynucleotides having the nucleic acid sequences of SEQ ID NOs: 3-18 and the complements the polynucleotides. The polynucleotides have been identified using the methods presented above, and the expression profiles for SEQ ID NOs: 3 and 18 produced using transcript imaging and presented in EXAMPLE VII confirm significant, tissue-specific, expression of these polynucleotides and the proteins or peptides they encode in kidney function or kidney disorders. In another embodiment, the invention encompasses methods that use the combination or individual polynucleotides selected from the combination.

The polynucleotide or its encoded protein or peptide can be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403-410), BLOCKS (Henikoff an Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp 856-853).

Also encompassed by the invention are polynucleotides that are capable of hybridizing to SEQ ID NOs: 3-18. Conditions for hybridization (e.g., Ausubel, supra, unit 2 pp. 1-41 and unit 4, pp. 22-27) can be selected by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5×SSC (saline sodium citrate) with 1% sodium dodecyl sulfate (SDS) at 60C, which permits complex formation between two nucleic acid sequences that contain some mismatches. Subsequent washes are performed at higher stringency with buffers such as 0.2×SSC with 0.1% SDS at either 45C (medium stringency) or 68C (high stringency), to maintain hybridization of only those complexes that contain completely complementary sequences. Background signals can be reduced by the use of detergents such as SDS, sarcosyl, or TRITON X-100 (Sigma-Aldrich, St. Louis Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning, A Laboratory Manual, Cold Spring Harbor Press, Plainview N.Y.)

A polynucleotide can be extended utilizing a partial nucleotide sequence and employing various methods such as PCR and shotgun cloning which are well known in the art. These methods can be used to extend upstream or downstream to obtain a full length sequence or to recover useful untranslated regions (UTRs), such as promoters and other regulatory elements. For PCR extensions, an XL-PCR kit (Applied Biosystems (ABI), Foster City Calif.), nested primers, and commercially available cDNA libraries (Invitrogen, Carlsbad Calif.) or genomic libraries (Clontech, Palo Alto Calif.) can be used to extend the sequence. For all PCR-based methods, primers can be designed using commercially available software to be about 15 to 30 nucleotides in length, to have a GC content of about 50%, and to form a hybridization complex at temperatures of about 68C to 72C.

In another aspect of the invention, the polynucleotide can be cloned into a recombinant vector that directs the expression of the protein, peptide, or structural or functional portions thereof, in host cells. Due to the inherent degeneracy of the genetic code, other DNA sequences which encode substantially the same or a functionally equivalent amino acid sequence can be produced and used to express the protein encoded by the polynucleotide. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides can be used to engineer the nucleotide sequences. For example, oligonucleotide-mediated site-directed mutagenesis can be used to introduce mutations that create new restriction sites, alter glycosylation patterns, change codon preference, produce splice variants, and so forth.

In order to express a biologically active protein, the polynucleotide or derivatives thereof, can be inserted into an expression vector which contains the elements for transcriptional and translational control of the inserted coding sequence in a particular host. These elements can include regulatory sequences, such as enhancers, constitutive and inducible promoters, and 5′ and 3′ untranslated regions. Methods which are well known to those skilled in the art can be used to construct such expression vectors. These methods include in vitro recombinant DNA techniques, synthetic techniques, and in vivo genetic recombination (Sambrook, supra; Ausubel, supra).

A variety of expression vector/host cell systems can be utilized to express the polynucleotide. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the polynucleotide can be transformed into cell lines using expression vectors which can contain viral origins of replication and/or endogenous expression elements and a selectable or visible marker gene on the same or on a separate vector. The invention is not to be limited by the vector or host cell employed.

In general, host cells that contain the polynucleotide and that express the protein can be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the polynucleotide can be cultured under conditions for the expression and recovery of the protein from cell culture. The protein produced by a transgenic cell can be secreted or retained intracellularly depending on the sequence and/or the vector used. As will be understood by those of skill in the art, expression vectors containing the polynucleotide can be designed to contain signal sequences which direct secretion of the protein through a prokaryotic or eukaryotic cell membrane.

In addition, a host cell strain can be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein can also be used to specify protein targeting, folding, and/or activity. Different host cells which have specific cellular machinery and characteristic mechanisms for post-translational activities (e.g., CHO, HeLa, MDCK, HEK293, and W138) are available from the ATCC (Manassas Va.) and can be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the polynucleotides, wholly or in part, are synthesized using chemical or enzymatic methods well known in the art (Caruthers et al. (1980) Nucl Acids Symp Ser (7) 215-233; Ausubel, supra). For example, peptide synthesis can be performed using various solid-phase techniques (Roberge et al. (1995) Science 269:202-204), and machines such as the 431A peptide synthesizer (ABI) can be used to automate synthesis. If desired, the amino acid sequence can be altered during synthesis and/or combined with sequences from other proteins to produce a variant.

Screening, Diagnostics and Thearpeutics

The polynucleotides are particularly useful as markers of kidney function and in diagnosis, prognosis, treatment, and selection and evaluation of therapies for kidney disorders. The polynucleotides can also be used to screen a plurality of molecules for specific binding affinity. The assay can be used to screen a plurality of DNA molecules, RNA molecules, peptide nucleic acids, peptides, ribozymes, antibodies, agonists, antagonists, immunoglobulins, inhibitors, proteins including transcription factors, enhancers, repressors, and drugs and the like which regulate the activity of the polynucleotide in the biological system. An exemplary assay involves providing a plurality of molecules, contacting the combination, the polynucleotide or a composition thereof, with the plurality of molecules under conditions to allow specific binding, and detecting specific binding to identify at least one molecule which specifically binds the polynucleotide.

Similarly proteins or peptides can be used to screen libraries of molecules or compounds in any of a variety of screening assays. The protein or peptide employed in such screening can be free in solution, affixed to an abiotic or biotic substrate (e.g. borne on a cell surface), or located intracellularly. Specific binding between the protein and the molecule can be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the polynucleotides are used for diagnostic purposes to determine the absence, presence, or differential expression. Differential expression must be increased or decreased as compared to a standard that is selected from either control cells, normal tissue, or well characterized diseased tissue. The polynucleotide consists of complementary RNA and DNA molecules, branched nucleic acids, and/or PNAs. In one alternative, the polynucleotides are used to detect and quantify gene expression in samples in which expression of the polynucleotide is indicative of kidney disorders. In another alternative, the polynucleotide can be used to detect genetic polymorphisms associated with kidney disorders. These polymorphisms can be detected in transcripts or genomic sequences.

The specificity of the probe is determined by whether it is made from a unique region, a regulatory region, or from a conserved motif. Both probe specificity and the stringency of hybridization or amplification (maximal, high, intermediate, or low) will determine whether the probe identifies only naturally occurring, exactly complementary sequences, allelic variants, or related sequences. Probes designed to detect related sequences should have at least 50% sequence identity and to detect a sequence having a polymorphism preferably 94% sequence identity.

Methods for producing hybridization probes include the cloning of the polynucleotide into vectors for the production of RNA probes. Such vectors are known in the art, are commercially available, and can be used to synthesize RNA probes in vitro by adding RNA polymerases and labeled nucleotides. Hybridization probes can incorporate nucleotides labeled by a variety of reporter groups including, but not limited to, radionuclides such as ³²P or ³⁵S, enzymatic labels such as alkaline phosphatase coupled to the probe via avidin/biotin coupling systems, fluorescent labels, and the like. The labeled polynucleotides can be used in Southern or northern analysis, dot or slot blot, or other membrane-based technologies; in PCR technologies; and in microarrays utilizing samples from subjects to detect differential expression.

The polynucleotide can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of differential expression in the sample indicates the presence of the disease. Qualitative and quantitative methods for comparing the hybridization complexes formed in subject samples with previously established standards are well known in the art.

Such assays can also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the subjects begins to approximate that which is observed in a healthy subject. The results obtained from successive assays can be used to show the efficacy of treatment over a period ranging from several days to many years.

The polynucleotides can be used as a combination or individually to assess kidney function or for the diagnosis of kidney disorders. The polynucleotides can also be used on a substrate such as microarray to monitor the expression patterns. The microarray can also be used to identify splice variants, mutations, and polymorphisms. Information derived from analyses of the expression patterns can be used to determine gene function, to understand the genetic basis of a disease, to diagnose a disease, and to develop and monitor the activities of therapeutic agents used to treat a disease. Microarrays can also be used to detect genetic diversity, single nucleotide polymorphisms which can characterize a particular population, at the genome level.

In yet another alternative, polynucleotides can be used to generate hybridization probes useful in mapping the naturally occurring genomic sequence. Fluorescent in situ hybridization (FISH) can be correlated with other physical chromosome mapping techniques and genetic map data as described in Heinz-Ulrich et al. (In: Meyers, supra, pp. 965-968).

In another embodiment, antibodies or Fabs comprising an antigen binding site that specifically binds the protein can be used for the diagnosis of diseases characterized by the over-or-under expression of the protein. A variety of protocols for measuring protein expression including ELISAs, RIAs, FACS, or arrays are well known in the art and provide a basis for diagnosing differential, altered or abnormal levels of expression. Standard values for protein expression are established by combining samples taken from healthy subjects, preferably human, with antibody to the protein under conditions for complex formation. The amount of complex formation can be quantitated by various methods, preferably by photometric means. Quantities of the protein expressed in disease samples are compared with standard values. Deviation between standard and subject values establishes the parameters for diagnosing or monitoring disease. Alternatively, one can use competitive drug screening assays in which neutralizing antibodies capable of binding specifically with the protein compete with a test compound. Antibodies can be used to detect the presence of any peptide which shares one or more antigenic determinants with the protein. In one aspect, the antibodies of the present invention can be used for treatment or monitoring therapeutic treatment for kidney disorders.

Recently, antibody arrays have allowed the development of techniques for high-throughput screening using recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. (See de Wildt et al. (2000) Nat Biotechnol 18:989-94.)

In another aspect, the polynucleotide, or its complement, can be used therapeutically for the purpose of expressing mRNA and protein, or conversely to block transcription or translation of the mRNA. Expression vectors can be constructed using elements from retroviruses, adenoviruses, herpes or vaccinia viruses, or bacterial plasmids, and the like. These vectors can be used for delivery of nucleotide sequences to a particular target organ, tissue, or cell population. Methods well known to those skilled in the art can be used to construct vectors to express nucleic acid sequences or their complements (see, e.g., Maulik et al. (1997) Molecular Biotechnology, Therapeutic Applications and Strategies, Wiley-Liss, New York N.Y.). Alternatively, the polynucleotide or its complement, can be used for somatic cell or stem cell gene therapy. Vectors can be introduced in vivo, in vitro, and ex vivo. For ex vivo therapy, vectors are introduced into stem cells taken from the subject, and the resulting transgenic cells are clonally propagated for autologous transplant back into that same subject. Delivery of the polynucleotide by transfection, liposome injections, or polycationic amino polymers can be achieved using methods which are well known in the art (See, e.g., Goldman et al. (1997) Nature Biotechnology 15:462-466). Additionally, endogenous gene expression can be inactivated using homologous recombination methods which insert an inactive gene sequence into the coding region or other targeted region of the polynucleotide (see, e.g. Thomas et al. (1987) Cell 51: 503-512).

Vectors containing the polynucleotide can be transformed into a cell or tissue to express a missing protein or to replace a nonfunctional protein. Similarly a vector constructed to express the complement of the polynucleotide can be transformed into a cell to downregulate the protein expression. Complementary or antisense sequences can consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (see, e.g., Gee et al. In: Huber and Carr (1994) Molecular and Immunologic Approaches, Futura Publishing, Mt. Kisco N.Y., pp. 163-177).

Ribozymes, enzymatic RNA molecules, can also be used to catalyze the cleavage of mRNA and decrease the levels of particular mRNAs, such as those comprising the polynucleotides of the invention (see, e.g., Rossi (1994) Current Biology 4: 469-47). Ribozymes can cleave mRNA at specific cleavage sites. Alternatively, ribozymes can cleave mRNAs at locations dictated by flanking regions that form complementary base pairs with the target mRNA. The construction and production of ribozymes is well known in the art and is described in Meyers (supra).

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, can be included.

Further, an agonist, an antagonist, or an antibody that binds specifically to the protein and modulates its activity can be administered to a subject to treat kidney disorders. The agonist, antagonist, or antibody can be used directly to enhance or inhibit the activity of the protein or indirectly to deliver a therapeutic agent to cells or tissues which express the protein. The therapeutic agent can be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein can be generated using methods that are well known in the art. The protein can be used to screen libraries or a plurality of antibodies to identify an antibody that specifically binds the protein. The antibody may be a polyclonal antibody, a monoclonal antibody, a chimeric antibody, a recombinant antibody, a humanized antibody, single chain antibodies, a Fab fragment, an F(ab′)₂ fragment, an Fv fragment; or an antibody-peptide fusion protein. Neutralizing antibodies, such as those which inhibit dimer formation, are especially preferred for therapeutic use. Monoclonal antibodies to the protein can be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used (see, e.g., Pound (1998) Immunochemical Protocols, Methods Mol Biol Vol. 80). Alternatively, techniques described for the production of single chain antibodies can be employed. Fabs which contain specific binding sites for the protein can also be generated. Various immunoassays can be used to identify antibodies having the desired specificity. Numerous protocols for competitive binding or immunoradiometric assays using either polyclonal or monoclonal antibodies with established specificities are well known in the art.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature and generally available to practitioners.

Further details on techniques for formulation and administration may be found in the latest edition of Remington's Pharmaceutical Sciences (Mack Publishing, Easton Pa.).

Stem Cells and Their Use

SEQ ID NOs: 3-18 can be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells can be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells can be transformed with polynucleotides which can be transiently expressed or can be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, can also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells can be used to treat neurological disorders such as Alzheimer disease, Parkinson disease, and multiple sclerosis and to repair tissue damaged by strokes and spinal cord injuries. Hematopoietic stem cells can be used to restore immune function in immunodeficient subjects or to treat autoimmune disorders by replacing autoreactive immune cells with normal cells to treat diseases such as multiple sclerosis, scleroderma, rheumatoid arthritis, and systemic lupus erythematosus. Mesenchymal stem cells can be used to repair tendons or to regenerate cartilage to treat arthritis. Liver stem cells can be used to repair liver damage. Pancreatic stem cells can be used to replace islet cells to treat diabetes. Muscle stem cells can be used to regenerate muscle to treat muscular dystrophies. (See, e.g., Fontes and Thomson (1999) BMJ 319:1-3; Weissman (2000) Science 287:1442-1446; Marshall (2000) Science 287:1419-1421; Marmont (2000) Ann Rev Med 51:115-134.)

### EXAMPLES

It is to be understood that this invention is not limited to the particular devices, machines, materials and methods described. Although equivalent embodiments can be used to practice the invention, the particular described embodiments were used and are not intended to limit the scope of the invention which is limited only by the appended claims.

I cDNA Library Construction

RNA was purchased from Clontech or isolated from kidney tissues, some of which are described for their polynucleotide expression in Example VII below. Some tissues were homogenized and lysed in guanidinium isothiocyanate; others were homogenized and lysed in phenol or a suitable mixture of denaturants, such as TRIZOL reagent (Invitrogen). The resulting lysates were centrifuged over CsCl cushions or extracted with chloroform. RNA was precipitated from the lysates with either isopropanol or sodium acetate and ethanol, or by other routine methods. Phenol extraction and precipitation of RNA were repeated as necessary to increase RNA purity.

In some cases, RNA was treated with DNAse. For most libraries, poly(A+) RNA was isolated using oligo d(T)-coupled paramagnetic particles (Promega, Madison Wis.), OLIGOTEX latex particles (Qiagen, Valencia Calif.), or an OLIGOTEX mRNA purification kit (Qiagen). Alternatively, RNA was isolated directly from tissue lysates using RNA isolation kits such as the POLY(A)PURE mRNA purification kit; Ambion, Austin Tex.).

In some cases, Stratagene (La Jolla Calif.) was provided with RNA and constructed the cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6). Reverse transcription was initiated using oligo d(T) or random primers. Synthetic oligonucleotide adapters were ligated to double stranded cDNA, and the cDNA was digested with the appropriate restriction enzyme(s). For most libraries, the cDNA was size-selected (300-1000 bp) using SEPHACRYL S1000, SEPHAROSE CL2B, or SEPHAROSE CL4B column chromatography (Amersham Pharmacia Biotech (APB), Piscataway N.J.) or preparative agarose gel electrophoresis. cDNAs were ligated into compatible restriction enzyme sites of the polylinker of pBLUESCRIPT plasmid (Stratagene), pSPORT1 plasmid (Invitrogen), or pINCY (Incyte Genomics). Recombinant plasmids were transformed into competent E. coli cells including XL1-BLUE, XL1-BLUEMRF, or SOLR (Stratagene) or DH5α, DH10β, or ElectroMAX DH10B (Invitrogen).

II Isolation, Sequencing and Analysis of cDNA Clones,

Plasmids were recovered from host cells by either in vivo excision using the UNIZAP vector system (Stratagene) or cell lysis. Plasmids were purified using one of the following kits or systems: a Magic or WIZARD Minipreps DNA purification system (Promega); an AGTC Miniprep purification kit (Edge Biosystems, Gaithersburg Md.); and QIAWELL 8 plasmid, QIAWELL 8 Plus plasmid, QIAWELL 8 Ultra Plasmid purification systems or the REAL Prep 96 plasmid kit (Qiagen). Following precipitation, plasmids were resuspended in 0.1 ml of distilled water and stored, with or without lyophilization, at 4C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a Fluoroskan II fluorescence scanner (Labsystems Oy, Helsinki, Finland).

The cDNAs were prepared for sequencing using the CATALYST 800 preparation system (ABI) or the HYDRA microdispenser (Robbins Scientific) or MICROLAB 2200 system (Hamilton, Reno Nev.) systems in combination with the DNA ENGINE thermal cyclers (MJ Research, Watertown Mass.). The cDNAs were sequenced using the PRISM 373 or 377 sequencing systems (ABI) and standard ABI protocols, base calling software, and kits. In one alternative, cDNAs were sequenced using the MEGABACE 1000 DNA sequencing system (Molecular Dynamics). In another alternative, the cDNAs were amplified and sequenced using the PRISM BIGDYE Terminator cycle sequencing ready reaction kit (ABI). In yet another alternative, cDNAs were sequenced using solutions and dyes from APB. Reading frames for the ESTs were determined using standard methods (reviewed in Ausubel, supra, unit 7.7).

The polynucleotide sequences derived from cDNA, extension, and shotgun sequencing were assembled and analyzed using a combination of software programs which utilize algorithms well known to those skilled in the art (Meyers, supra, pp 856-853).

III Assembly of Polynucleotides and Characterization of Sequences

The sequences used for co-expression analysis were assembled from EST sequences, 5′ and 3′ long read sequences, and full length coding sequences.

The polynucleotides of this application were compared with assembled consensus sequences or templates found in the LIFESEQ GOLD database (Incyte Genomics). Component sequences from polynucleotide, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms (Incyte Genomics) that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of <1×10⁻⁸. The templates were also subjected to frameshift FASTx against GENPEPT, and homolog match was defined as having an E-value of <1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed October 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.).

The BLAST software suite, freely available sequence comparison algorithms (NCBI, Bethesda Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to idenitify structural homologs by sequence identity and found 30% identity is a reliable threshold for sequence alignments of at least 150 residues and 40%, for alignments of at least 70 residues.

The polynucleotide and any encoded protein were further queried against public databases such as the GenBank rodent, mammalian, vertebrate, prokaryote, and eukaryote databases, SwissProt, BLOCKS, PRINTS, PFAM, and Prosite.

IV Expression of Polynucleotides in Kidney

Known Genes Expressed with High Specificity in Kidney

There are 19 known genes that are expressed with very high specificity in kidney. These genes, their Incyte Gene I), GenBank designation, name, cell location and p-value (using the Fisher Exact Test) are shown in the table below.

| Gene ID | GenBank | Name | Cell location | P-value |
|---|---|---|---|---|
| 361108 | g340165 | Uromodulin (Tamm- | TAHL | 3.1e-28 |
| | | Horsfall glycoprotein, | | |
| | | THG) | | |
| 209467 | g3523100 | Ksp-cadherin (CDH16) | BBM/* | 4.3e-25 |
| 332054 | g1373424 | Bumetanide-sensitive | BBM/TALH | 4.3e-24 |
| | | Na—K—2Cl cotrans- | | |
| | | porter (NKCC2) | | |
| 333342 | g1172160 | Thiazide-sensitive | BBM/DCT | 9.8e-18 |
| | | Na—Cl cotransporter | | |
| | | (TSC) | | |
| 429891 | g433142 | Inwardly rectifying K+ | BBM/TALH | 1.0e-13 |
| | | channel (ROMK1) | | |
| 336259 | g292349 | Renal Na/Pi-cotrans- | BBM/PCT | 5.3e-13 |
| | | porter (NaPi-IIa) | | |
| 334222 | g4378058 | Organic anion | | 4.3e-12 |
| | | transporter (OAT3) | | |
| 403619 | g7363001 | Podocin (NPHS2 gene) | SD/Podocyte | 1.6e-11 |
| 344395 | g2062691 | Sodium phosphate | | 1.6e-11 |
| | | transporter (NPT4) | | |
| 343903 | g35951 | Renin | JGA | 2.6e-11 |
| 344760 | g2281941 | Organic cation trans- | BLM/PCT/ | 9.5e-11 |
| | | porter, kidney (OCT2) | BBM/DCT | |
| 334445 | g639841 | Renal Na+-dependent | BBM/PCT | 4.4e-10 |
| | | phosphate cotransporter | | |
| | | (NPT1) | | |
| 161090 | g4502184 | Aquaporin 6 (AQP6, or | ICMV/* | 1.8e-9 |
| | | Kidney water channel, | | |
| | | KID) | | |
| 229645 | g6009532 | Tubulointerstitial | EM/* | 1.8e-8 |
| | | nephritis antigen | | |
| | | (TIN-ag) | | |
| 247379 | g4579724 | Organic anion transporter | BBM/PCT | 1.8e-8 |
| | | (OAT1) | | |
| 251820 | g9992883 | Vacuolar proton pump | BBM/CD | 2.9e-7 |
| | | 116 kDa accessory | | |
| | | subunit | | |
| 404129 | g3025698 | Nephrin (NPHS1) | Podocyte | 3.2e-7 |
| 228177 | g6651445 | Putative N-acetyl- | | 6.6e-13 |
| | | transferase CML1 | | |
| 897901 | g9957753 | Kidney-specific | | 1.3e-9 |
| | | membrane protein NX-17 | | |

Abbreviations: BBM, brush-border membrane; BLM, basolateral membrane; SD, slit diaphragm; ICMV, intracelluar membrane vesicle; EM, extracellular matrix; JGA, juxtaglomerular apparatus; TALH, thick ascending limb of Henle's loop; PCT, proximal convoluted tubule; DCT, distal convoluted tubule; CD, collecting duct; and *, all tubular segments of nephron and the collecting duct system.

Most of the known genes have been categorized both for their function in glomerular filtration, tubular reabsorption/excretion, matrix remodeling,, renin-angiotensin system, or immunomodulation and for their role in kidney function or disorders. A short description for each protein and it encoding gene are presented below.

Glomerular Filtration

Nephrin (NPHS1; g3025698) is a central component of the podocyte slit diaphragm, is essential for the normal renal filtration (Kestila, supra), and has a predicted extracellular domain and single transmembrane span typical of a cell adhesion molecule. The gene that encodes nephrin is mutated in congenital nephrotic syndrome (MIM 256300).

Podocin (NPHS2; g7363001) is almost exclusively expressed in the podocytes of fetal and mature kidney glomeruli and encodes an integral membrane protein that belongs to the stomatin protein family. Podocin is the protein/gene that causes autosomal recessive steroid-resistant nephrotic syndrome (MIM 600995; Boute et al. (2000) Nature Genet 24:349-354 [published erratum: Nature Genet 25:125]).

Tubular Reabsorption/Secretion

Bumetanide-sensitive Na—K-2Cl cotransporter (NKCC2; g1373424) is expressed in the apical membrane of the epithelial cells of the thick ascending limb of Henle's loop (TALH) and of the macula densa, accounts for almost all luminal NaCl reabsorption in the TALH, and is a member of a diverse family of cation (Na/K)-chloride cotransport proteins that share a common predicted membrane topology. The transport process is characterized by electroneutrality, affected by a large variety of hormonal stimuli as well as by changes in cell volume, and inhibited by “loop” diuretics—bumetanide, benzmetanide, and furosemide. Genetic mutations result in Bartter syndrome (MIM 600839; Simon et al. (1996) Nature Genet 13:183-188).

Thiazide-sensitive Na—Cl cotransporter (TSC; g1172160) is expressed in the apical membrane of distal convoluted tubule (DCT) cells, where the majority of Na+ and Cl— are reabsorbed, contains 12 membrane-spanning domains, and is oriented with the amino- and carboxyl-termini within the cytoplasm. Genetic mutations have been shown to cause Gitelman syndrome (MIM 263800: Mastroianni et al. (1996) Genomics 35:486-493; Simon et al. (1996) Nature Genet 12:24-30).

Inwardly rectifying K+ channel (ROMK1; g433142) which belongs to a family of that same name characterized by little-to-no voltage dependence, inward rectification, exquisite pH-sensitivity, and modulation by ATP and is involved in potassium recycling secretion in the TALH and potassium secretion in cortical collecting duct (Kohda et al. (1998) Kidney Int 54:1214-1223). In human kidney, differential splicing produces five distinct transcripts of ROMK1, all of which contain exon 5 that encodes the majority of the protein. Genetic mutations cause the antenatal variant of Bartter syndrome (MIM 600359; Derst et al. (1997) Biochem Biophys Res Commun 230:641-5).

Renal Na/Pi-cotransporter (NaPi-Ila, NPT2, NAPI-3; g292349) is expressed in the apical membrane 30 of proximal convoluted tubule (PCT) cells to control overall Pi homeostasis in the renal proximal tubule (Murer et al. (2000) Physiol Rev 80:1373-409). Protein expression is also affected by hormonal and metabolic factors known to influence extracellular fluid Pi homeostasis (Karim-Jimenez et al. (2000) Proc Natl Acad Sci 97:12896-901).

Renal Na+-dependent phosphate cotransporter (NaPi-1, NPT1, NaPi-4, SLC17A1; g639841) appears to be a multifunctional anion channel protein with expression in renal brush-border membrane and permeability for chloride and different organic anions (Uchino et al. (2000)Antimicrob Agents Chemother 44:574-7).

Sodium phosphate transporter (NPT4, SLC17A3; g2062691) is mapped 0.1 Mb centromeric to the gene encoding NPT1 and is one of the two genes cloned from the hereditary hemochromatosis locus which show indistinguishable hydrophobicity profiles from and appreciable homology to NPT1 (Ruddy et al. (1997) Genome Res 7:441-56).

Organic cation transporter (OCT2; g2281941) is localized at the luminal membrane of the distal 10 convoluted tubule (Urakami et al. (1998) J Pharmacol Exp Ther 287:800-805) where it has an affinity for various positively charged organic solutes (xenobiotics, metabolites, and drugs) and also accepts dopamine and other monoamine transmitters as substrate (Grundemann et al. (1998) J Biol Chem 273:30915-20).

Multispecific organic anion transporter 1 (OAT1; g4579724) mediates transport of endogenous or environmental anions with different chemical structures and a number of clinically important anionic drugs across the basolateral membrane of the renal proximal tubule. Multispecific organic anion transporter 3 (OAT3; g4378058), which is expressed strongly in kidney, also mediates the coupled exchange of alpha-ketoglutarate with multiple organic anions, including p-aminohippurate. Both OAT1 and OAT3 map to chromosome 11 region q11.7 (Race et al. (1999) Biochem Biophys Res Commun 255:508-514).

Aquaporin 6 (AQP6, hKID; g4502184), a member of the aquaporin family (Yasui et al. (1999) Proc Natl Acad Sci 96:5808-5813), is present in membrane vesicles within podocyte cell bodies and foot processes and within the subapical compartment of segment 2 and segment 3 cells in proximal tubules and in intracellular vesicles of the apical, mid, and basolateral cytoplasm of type A intercalated cells of the collecting duct. Its unique distribution in intracellular membrane vesicles in multiple types of renal epithelia indicates that AQP6 has a wider role than transcellular fluid absorption (Yasui et al. (1999) Nature 402:184-187).

Vacuolar proton pump 116 kDa accessory subunit (ATP6N1A; g9992883), which is hydrophilic and likely to be intracellular, localizes almost exclusively and at particularly high density on the apical (luminal) surface of alpha-intercalated cells of the cortical collecting duct of the distal nephron where vectorial proton transport is required for urinary acidification. Genetic mutations in the gene cause renal tubule acidosis accompanied by deafness (MIM 267300).

Matrix and adhesion proteins

Tubulointerstitial nephritis antigen (TIN-ag; g6009532) has a cysteine-rich follistatin module, six potential glycosylation sites, and an ATP/GTP-binding site and is homologous to several classes of extracellular matrix molecules in its amino terminal region and to cathepsin family of cysteine proteinases in its carboxyl terminal region. TIN-ag is an extracellular matrix basement protein originally identified as a target antigen involved in anti-tubular basement membrane antibody-mediated interstitial nephritis (Katz et al. (1992) Am J Med 93:691-698). which plays a role in renal tubulogenesis and has been implicated in hereditary tubulointerstitial disorder, particularly juvenile nephronophthisis (Nelson et al. (1998) Connect Tissue Res 37:53-60; Ikeda et al. (2000) Biochem Biophys Res Commun 268:225-230).

Ksp-cadherin (CDH16; g3523100) is a kidney-specific membrane-associated glycoprotein of the cadherin superfamily of cell adhesion molecules (Thomson et al. (1998) Genomics 51:445-451) which mediate Ca2+-dependent cellular recognition and adhesion and are thought to play an integral role in both tissue morphogenesis and maintenance of the differentiated phenotype. Ksp-cadherin is expressed on the basolateral surface of all tubular segments of the nephron and the collecting duct system.

Renin-Angiotensin System

Renin (REN; g35951) is an aspartyl protease, released by kidney cells (juxtaglomerular apparatus) when renal blood pressure or oxygen levels decline, that cleaves angiotensinogen to produce angiotensin II. which in turn increases blood pressure.

Immunomodulator

Uromodulin (TBP; g340165), the most abundant glycoprotein in mammalian urine, is known for its ability to suppress antigen-induced proliferation of peripheral blood mononuclear cells by binding proinflammatory cytokines and inhibiting in vitro T cell proliferation induced by specific antigens (Muchmore and Decker (1985) Science 229:479-481, Hession et al. (1987) Science 237:1479-1484, and Su and Yeh (1999) Life Sci 65:2581-2590). THP has been implicated in maintenance of electrolyte balance in the nephron and is thought to protect the kidneys from bacterial infections and to play a significant role in acute renal failure, urinary tract infection, stone formation, and interstitial nephritis (Easton et al. (2000) J Biol Chem 275:21928-38).

V Kidney Function and Kidney Disorder Specific Polynucleotides

Using the data in the LIFESEQ GOLD database (release October 2000; Incyte Genomics), 16 polynucleotides that showed highly significant expression, a cutoff p-value of less than 0.00001 (P<1e⁻⁵), in kidney or kidney disorders were identified. The statistical method presented in the DESCRIPTION OF THE INVENTION was used to identify these polynucleotides among approximately five million cDNAs assigned to one of the 40,285 gene bins. The table below shows the expression of polynucleotides (Incyte ID) that match unannotated public sequences.

| Incyte ID | GenBank | Name | P-value |
|---|---|---|---|
| 337832 | g7020765 | FLJ20569 fis, clone REC00864 | 6.2e-12 |
| 332290 | g7022812 | FLJ10650 fis, clone NT2RP2005853 | 5.2e-10 |

Incyte ID 337832 matches the first 1084 nucleotides of a public sequence, g7020765, containing 1166 nucleotides that encodes a hypothetical protein homologous to mouse kidney aldehyde reductase 6. A single base insertion (C522) also occurs in the alignment of 337832.3 with a genomic sequence g5804920 from clone 579N16 on chromosome 22 that is 66,618 nucleotides in length.

Incyte ID 332290 matches the first 435 nucleotides of g7022812 which aligns with genomic sequence g12001742 (chromosome 14 clone R-409I10 that is 151,879 nucleotides in length).

Polynucleotides with Known Homologs

BLAST analysis identified four polynucleotides, shown in the table below, with sequence identity to known genes from human, rat, or mouse. In particular, Incyte ID 210710 encodes a novel human organic anion transporter protein with homology to mouse RST, an organic cation transporter (Mori et al. (1997) FEBS Lett 417:371-374).

| Gene ID | Species | GenBank | Name | P-value |
|---|---|---|---|---|
| 279978 | Rat | 3127193 | Kidney-specific protein (KS) | 1.1e-23 |
| 210710 | Mouse | 2696709 | Renal-specific transporter | 4.4e-10 |
| | | | (RST) | |
| 134574 | Human | 10435135 | FLJ13212 fis, clone | 5.4e-8 |
| | | | NT2RP4001029 | |
| 400839 | Mouse | 951098 | Nuclear factor NF2d9 | 7.2e-8 |

The closest homolog to Incyte ID 279978 is g=3127193, a rat kidney-specific protein. SEQ ID NO: 17 encodes the polypeptide of SEQ ID NO: 1 which is 577 amino acids in length and displays 77% sequence identity to rat protein (Hilgers et al. (1998) Kidney Int 54:1444-1454), 57% identity to the hypertension related SA gene product (Samani and Lodwick (1995) J Hum Hypertens 9:501-503), and approximately 50% similarity to prokaryotic and eukaryotic acetyl-CoA synthases. Part of SEQ ID NO: 17 matches genomic sequence from chromosome 16 BAC clone CIT987SK-A-923A4 (g3219338) which is spliced into 8 exons; however, g3219338 misses an unknown number of 5′ exons, and a smaller protein (207 residues) which has been annotated as “homolog of rat kidney-specific gene” corresponds to the C-terminal half of SEQ ID NO: 1.

The closest homolog to Incyte ID 210710 is g2696709, mouse renal-specific transporter (RST). SEQ ID NO: 2 is 74% identical to mouse RST at the amino acid level. Mouse RST is a novel 12 membrane-spanning transporter like-protein (Mori, sura) whose expression is restricted to the renal proximal tubule. Although mouse RST was predicted to be an organic cation transporter based on its 30% identity to the type 1 rat organic cation transporter, SEQ ID NO: 2 shows that the translated polypeptide of 210710 exhibits 53% sequence identity with human organic anion transporter 4 (hOAT4).

### VI Novel Kidney-specific Polynucleotides

Novel kidney-specific polynucleotides are shown in the table below. The first column shows the Incyte ID of the polynucleotide; the second column, the P-value; the third column, the chromosomal location of the poynucleotide, the fourth column, the genomic sequence that has exons that match the polynucleotide; and the fifth column, identification of a nearby gene or Incyte ID. The table is subdivided into those polynucleotides that are adjacent to other known genes, those that match an intron, those that match known genomic sequence and those that have no known match.

| | | | | |
|---|---|---|---|---|
| Incyte | | | Genomic | |
| ID | P-value | Chrm | sequence | Nearby gene or Incyte ID |

Polynucleotides that are adjacent to other known genes

| | | | | |
|---|---|---|---|---|
| 4516 | 1.8e-12 | 7 | g8887028 | g9992883 (Incyte 251825) |
| 213764 | 1.8e-9 | 7 | g8887028 | g9992883 (Incyte 251825) |
| 249553 | 7.9e-10 | 16 | g3219338 | Incyte: 279978 |
| 413721 | 3.2e-7 | 16 | g3219338 | Incyte: 279978 |
| 345462 | 1.4e-7 | 5 | g8698772 | g7019811 (Incyte 1398404) |

Polynucleotides that match the intron of a known gene

| | | | | |
|---|---|---|---|---|
| 108833 | 1.8e-9 | 14 | g12001742 | g7022812 (Incyte: 197930.31, |
| | | | | 332290.1) |
| 393706 | 5.4e-8 | 17 | g3126781 | Incyte: 1100433 and 407063 |

Polynucleotides that match known genomic sequence

| | | | |
|---|---|---|---|
| 4742 | 1.2e-8 | 7 | g11465194 |
| 980289 | 5.4e-8 | 5 | g7709149 |
| 311180 | 5.4e-8 | 5 | g6778453 |
| 334440 | 3.3e-7 | 19 | g11119455 |

Polynucleotides that have no known match

| | |
|---|---|
| 71972 | 5.4e-8 |
| 71870 | 5.4e-8 |
| 405479 | 3.2e-7 |

VII Co-expression of Genes and Polynucleotides Specific for Kidney or Kidney Disorders

The table below shows the co-expression of the known kidney genes with previously uncharacterized Incyte polynucleotides. Coexpression was measured using the GBA method described in Walker (supra). The table shows the probability (−log₁₀P) that the observed co-expression of any pair of genes (or polynucleotides) is due chance, as measured by the Fisher Exact Test. Cells with no entry represent P-values larger than 10e−3. Each of the polynucleotides was found to co-express with at least one known kidney-specific gene with P<10e−7. This result provides very strong evidence that the identified polynucleotides are truly kidney-specific.

| Incyte ID | 4516 | 213764 | 249553 | 345462 | 108833 | 393706 | 4742 | 980289 | 31118 | 334440 | 71972 | 71870 | 405479 |
|---|---|---|---|---|---|---|---|---|---|---|---|---|---|
| 361108 | 13 | 6.7 | 6.5 | | 7.3 | | 4.9 | | | | 8 | 8 | |
| 209467 | 8.7 | | 8.7 | | 7.6 | | | | | | 6 | 8.4 | |
| PB-0022 US | | | | | | | | | | | | | |
| 332054 | 14.1 | 7.3 | 7.2 | | | | | | | | 8.7 | 6.2 | |
| 333342 | 12.5 | 8.1 | 4.7 | | 6.3 | | | | 4.6 | | 9.4 | 6.8 | 4.3 |
| 429891 | 8.8 | | | | | | | | | | 7.7 | 5.2 | |
| 336259 | 9.1 | 7 | | | | | | | | | 7.8 | 7.8 | |
| 334222 | 10.7 | 4.4 | 8.9 | | | | | | 4.9 | | | | |
| 403619 | 4.7 | 7.2 | | | | | | | | | 8 | | |
| 344395 | | | 5.9 | | 5.2 | 5.2 | 7.2 | | | 4.4 | | | |
| 343903 | 7.3 | 10.1 | 6 | 8 | 6 | 8.3 | | 9.1 | 6.5 | | | | 6 |
| 344760 | 6.2 | | 5.2 | | 4.7 | | 8.9 | | 5 | | | | 6.9 |
| 334445 | | | | | | | 9.8 | | | 4.4 | | | |
| 161090 | 5.5 | | | | 3.6 | | | 3.7 | | 5.2 | 6.3 | 3.7 | |
| 229645 | | | | 5.1 | 6.6 | 9.1 | 8.5 | 7.1 | 7.1 | 3.7 | | | 4.5 |
| 247379 | | | 5 | | | | | | 4.8 | | | | |
| 404129 | | | | | | | | | | | 3.7 | | |
| 228177 | 7.2 | | 7.6 | | | | | | 4.3 | | | 4.3 | |
| 897901 | | 5.4 | 5.7 | | | | 9.7 | | | | 4.2 | | |

The results above are summarized in the following table which shows the known gene with which each polynucleotide is most closely co-expressed and the kidney function or disorder for which the polynucleotide serves as a surrogate marker.

| Incyte ID | Known Gene | Utility in Kidney (function or disorder) |
|---|---|---|
| 4516 | g1373424; NKCC2 | Bartter syndrome |
| 213764 | g35951; renin | control of blood pressure |
| 249553 | g4378058; OAT3 | drug clearance |
| 345462 | g35951; renin | control of blood pressure |
| 108833 | g3523100; CDK16 | maintenance of differentiated renal cells |
| 393706 | g6009532; TIN-AG | interstitial nephritis |
| 4742 | g639841; NPT1 | chloride and phosphate homeostasis |
| 980289 | g35951; renin | control of blood pressure |
| 311180 | g6009532; TIN-AG | interstitial nephritis |
| 334440 | g4502184; KID | control of blood pressure |
| 71972 | g1172160; TCS | Gitelman syndrome |
| 71870 | g3523100; CDK16 | maintenance of differentiated renal cells |
| 405479 | g2281941; OCT2 | xenobiotic, metabolite, and drug |
| | | clearance |

Transcript Imaging

The following transcript images demonstrate the specificity of polynucleotide expression in kidney and support the data produced using GBA. A transcript image was performed using the LIFESEQ GOLD database (Jan02release, Incyte Genomics). This process allowed assessment of the relative abundance of the expressed polynucleotides in all of the cDNA libraries and was described in U.S. Pat. No. 5,840,484, incorporated reference.

Criteria for transcript imaging were selected from category, number of cDNAs per library, library description, disease indication, clinical relevance of sample, and the like. Zweiger (2001) Transducing the Genome. McGraw Hill, San Francisco Calif.) and Glavas et al. (2001, Proc Natl Acad Sci 6319-6324), both incorporated herein by reference, discussed the time-delayed, close correspondence between most mRNA and protein expression.

All polynucleotides and cDNA libraries in the LIFESEQ database have been categorized by system, organ/tissue and cell type. For each category, the number of libraries in which the polynucleotide was expressed were counted and shown over the total number of libraries in that category. For each library, the number of cDNAs were counted and shown over the total number of cDNAs in that library. In some transcript images, all normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs such as heart, kidney, nerves or pancreas would be aided by removing clinical samples from the analysis.

The exemplary transcript images for SEQ ID NOs: 3 and 18 are shown in the tables below. The first table shows the expression of the polynucleotide among the categories in the LIFESEQ GOLD database. The first column shows category; the second column, the number of cDNAs sequenced in that category; the third column, the number of libraries in which the sequence was expressed over the total number of libraries in the category, the fourth column, absolute abundance of the transcript in the category; and the fifth column, percentage abundance of the transcript in the category

| | | | | |
|---|---|---|---|---|
| Category | cDNAs | #Libs | Abund | % Abund |

SEQ ID NO: 3 (Incyte ID 004516)

| | | | | |
|---|---|---|---|---|
| Cardiovascular | 278621 | 0/78 | 0 | 0.0000 |
| Connective Tissue | 151680 | 0/54 | 0 | 0.0000 |
| Digestive | 572415 | 0/164 | 0 | 0.0000 |
| Embryonic | 134983 | 0/30 | 0 | 0.0000 |
| Endocrine | 245132 | 0/73 | 0 | 0.0000 |
| Exocrine Glands | 298121 | 0/73 | 0 | 0.0000 |
| Female Reproductive | 486361 | 0/123 | 0 | 0.0000 |
| Male Reproductive | 489837 | 0/129 | 0 | 0.0000 |
| Germ Cells | 48479 | 0/5 | 0 | 0.0000 |
| Hemic/Immune | 764592 | 0/191 | 0 | 0.0000 |
| Liver | 142156 | 0/42 | 0 | 0.0000 |
| Musculoskeletal | 177848 | 0/54 | 0 | 0.0000 |
| Nervous | 1051758 | 0/239 | 0 | 0.0000 |
| Pancreas | 115806 | 0/27 | 0 | 0.0000 |
| Respiratory System | 442179 | 0/101 | 0 | 0.0000 |
| Sense Organs | 31671 | 0/12 | 0 | 0.0000 |
| Skin | 85255 | 0/19 | 0 | 0.0000 |
| Stomatognathic | 14930 | 0/20 | 0 | 0.0000 |
| Unclassified/Mixed | 200857 | 0/27 | 0 | 0.0000 |
| Urinary Tract | 321635 | 8/77 | 9 | 0.0028 |
| Totals | 6054316 | 8/1538 | 9 | 0.0001 |

SEQ ID NO: 18 (Incyte ID 210710)

| | | | | |
|---|---|---|---|---|
| Cardiovascular | 278621 | 0/78 | 0 | 0.0000 |
| Connective Tissue | 151680 | 0/54 | 0 | 0.0000 |
| Digestive | 572415 | 0/164 | 0 | 0.0000 |
| Embryonic Structures | 134983 | 0/30 | 0 | 0.0000 |
| Endocrine | 245132 | 0/73 | 0 | 0.0000 |
| Exocrine Glands | 298121 | 0/73 | 0 | 0.0000 |
| Female Reproductive | 486361 | 0/123 | 0 | 0.0000 |
| Male Reproductive | 489837 | 0/129 | 0 | 0.0000 |
| Germ Cells | 48479 | 0/5 | 0 | 0.0000 |
| Hemic/Immune | 764592 | 0/191 | 0 | 0.0000 |
| Liver | 142156 | 0/42 | 0 | 0.0000 |
| Musculoskeletal | 177848 | 0/54 | 0 | 0.0000 |
| Nervous System | 1051758 | 0/239 | 0 | 0.0000 |
| Pancreas | 115806 | 0/27 | 0 | 0.0000 |
| Respiratory | 442179 | 0/101 | 0 | 0.0000 |
| Sense Organs | 31671 | 0/12 | 0 | 0.0000 |
| Skin | 85255 | 0/19 | 0 | 0.0000 |
| Stomatognathic | 14930 | 0/20 | 0 | 0.0000 |
| Unclassified/Mixed | 200857 | 0/27 | 0 | 0.0000 |
| Urinary Tract | 321635 | 8/77 | 12 | 0.0037 |
| Totals | 6054316 | 8/1538 | 12 | 0.0000 |

The expression of SEQ ID NOs: 3 and 18 in the urinary tract are shown in the tables below. The first column shows library name; the second column, the number of cDNAs sequenced in that library; the third column, the description of the library; the fourth column, absolute abundance of the transcript in the library; and the fifth column, percentage abundance of the transcript in the library.

| | | | | |
|---|---|---|---|---|
| | | | Abun- | % Abun- |
| Library* | cDNAs | Description of Tissue | dance | dance |

SEQ ID NO: 3 (Incyte ID 004516)
Category: Urinary Tract (Kidney)

| | | | | |
|---|---|---|---|---|
| KIDCTMT02 | 1864 | kidney, cortex, mw/renal | 1 | 0.0536 |
| | | cell CA, 65M | | |
| KIDCTME01 | 3388 | kidney, cortex, mw/renal | 1 | 0.0295 |
| | | cell CA, 65M, 5RP | | |
| KIDNNOT25 | 3796 | kidney, mw/benign cyst, | 1 | 0.0263 |
| | | nepbrolithiasis, 42F | | |
| KIDCTMT01 | 6140 | kidney, cortex, mw/renal | 1 | 0.0163 |
| | | cell CA, 65M | | |
| KIDNNOT19 | 6949 | kidney, mw/renal cell | 1 | 0.0144 |
| | | CA, 65M, | | |
| | | m/KIDNTUT15 | | |

SEQ ID NO: 18 (Incyte ID 210710)
Category: Urinary Tract (Kidney)

| | | | | |
|---|---|---|---|---|
| KIDNNOT20 | 3709 | kidney, mw/renal cell | 2 | 0.0539 |
| | | CA, 43M, | | |
| | | m/KIDNTUT14 | | |
| KIDCTMT02 | 1864 | kidney, cortex, mw/renal | 1 | 0.0536 |
| | | cell CA, 65M | | |
| KIDNNOT32 | 5619 | kidney, 49M | 1 | 0.0178 |
| KIDCTMT01 | 6140 | kidney, cortex, mw/renal | 1 | 0.0163 |
| | | cell CA, 65M | | |

*Libraries made from subtracted tissue and from tissue identified only by markers have been removed from this analysis.
*Libraries made from normalized, subtracted, enriched, or pooled tissues have been removed from this analysis.

A summary of the expression for all of the polynucleotides and their support for GBA as summarized from TIs are shown below. The first column shows SEQ IN NO for the polynucleotide; the second column, the number of libraries in which the polynucleotide was expressed; the third column, the number of times the polynucleotide was expressed in kidney libraries; the fourth column, the percent specificity of expression; and the fifth column, other libraries in which the polynucleotide was expressed

| | | Amount | Specificity | Other |
|---|---|---|---|---|
| SEQ ID | Libraries* | Expression | (%) | Expression |
| 4 | 8 | 10 | 50 | liver |
| 5 | 6 | 10 | 91 | unclassified/mixed |
| 6 | 7 | 8 | 100 | |
| 7 | 7 | 7 | 78 | nervous |
| 8 | 6 | 9 | 90 | unclassified/mixed |
| 9 | 3 | 7 | 100 | |
| 10 | 5 | 8 | 100 | |
| 11 | 5 | 6 | 100 | |
| 12 | 6 | 7 | 100 | |
| 13 | 5 | 5 | 71 | unclassified/mixed |
| 14 | 5 | 6 | 86 | female |
| | | | | reproductive |
| 15 | 5 | 7 | 29 | liver |
| 16 | 7 | 9 | 70 | various |
| 17 | 12 | 21 | 58 | liver |

*48 kidney libraries are represented in the LIFESEQ GOLD database

Descriptions of Libraries Appearing in the TI

The KIDCTME01, KIDCTMT01 and KIDCTMT02 cDNA libraries were constructed using polyA RNA isolated from kidney tissue removed from a 65-year-old male during nephroureterectomy. Pathology indicated the margins of resection were free of involvement. Pathology for the associated tumor tissue Indicated grade 3 renal cell carcinoma, clear cell type, forming a variegated multicystic mass situated within the mid-portion of the kidney. The tumor invaded deeply into, but not through, the renal capsule; and the hilum (ureter, renal artery, and renal vein) and regional lymph nodes were free of involvement.

The KIDNNOT19 cDNA library was constructed using polyA RNA isolated from kidney tissue removed a 65-year-old Caucasian male during an exploratory laparotomy and nephroureterectomy. Pathology for the matched tumor tissue indicated a grade 1 renal cell carcinoma, clear cell type, forming a variegated mass situated within the upper pole of the left kidney. The overlying capsule was free of involvement. Five microscopically similar satellite tumor nodules were identified, the largest was situated four cm from the main tumor mass. The renal vein, artery, hilar lymph nodes, and ureter were free of involvement. The patient presented with abdominal pain, and patient history included a retinal hole, benign hypertension, malignant melanoma of the abdominal skin, benign neoplasm of colon, cerebrovascular disease, and umbilical hernia. Previous surgeries included blepharoplasty, umbilical hernia repair, rotator cuff repair, and vasectomy. Patient medications included verapamil hydrochloride, Zestril (lisinopril), aspirin, and garlic pills. Family history included myocardial infarction, atherosclerotic coronary artery disease, cerebrovascular disease, and prostate cancer.

The KIDNNOT20 cDNA library was constructed using polyA RNA isolated from left kidney tissue removed from a 43-year-old Caucasian male during nephroureterectomy, regional lymph node excision, and unilateral left adrenalectomy. Pathology for the matched tumor tissue indicated a grade 2 renal cell carcinoma forming a mass in the posterior lower pole of the left kidney with invasion into the renal pelvis. The tumor perforated the renal capsule into perinephric fat. The renal vein and ureteral and radial fat margins were free of tumor. The adrenal gland showed no diagnostic abnormalities, and multiple lymph nodes were negative for tumor. The patient was not taking any medications, but presented with deficiency anemia and hematuria. Patient history included benign hypertension and obesity and previous adenotonsillectomy and inguinal hernia repair. Family history included benign hypertension and atherosclerotic coronary artery disease.

The KIDNNOT25 cDNA library was constructed using polyA RNA isolated from kidney tissue removed from the left lower kidney pole of a 42-year-old Caucasian female during nephroureterectomy. Pathology for this sample was benign and for the matched diseased tissue, indicated benign simple cysts, slight hydronephrosis, and nephrolithiasis with stones of various sizes. The patient presented with calculus of the kidney, abnormal kidney function, and an unspecified congenital abnormality. Patient history included benign hypertension and kidney stones. Previous surgeries included an electroshock wave lithotripsy, and patient medications included Bicita, HCTZ, Allopurinor, Cephalexin, and Darvocet 100. Family history included benign hypertension and alcohol abuse.

The KIDNNOT32 cDNA library was constructed using polyA RNA isolated from kidney tissue removed from a 49-year-old Caucasian male who died from an intracranial hemorrhage and cerebrovascular accident. Serology was positive for anti-CMV, and patient history included tobacco abuse (2-½ packs per day) and alcohol use. Previous surgeries included an unspecified knee surgery and a vasectomy.

IX Hybridization Technologies and Analyses

Incyte clones represent template sequences or ESTs derived from the LIFESEQ GOLD assembled human sequence database (Incyte Genomics). In cases where more than one clone was available for a particular template, the 5′-most clone in the template was used on the microarray. The HUMAN GENOME GEM series 1-5 microarrays (Incyte Genomics) contain 45,320 array elements which represent 22,632 annotated clusters and 22,688 unannotated clusters. For the UNIGEM series microarrays (Incyte Genomics), Incyte clones were mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) was chosen, verified, and used in the construction of the microarray. The UNIGEM V 2.0 microarray (Incyte Genomics) contains 8,502 array elements which represent 8,372 annotated genes and 130 unannotated clusters.

Immobilization of Polvnucleotides on a Substrate

Polynucleotides are applied to a substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2×SSC for 10 min ea The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigrna-Aldrich) in 95% ethanol, and curing in a 110C oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for Membrane Hybridization

Hybridization probes derived from the polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100C for five min, and briefly centrifuging. The denatured polynucleotide is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for Polymer Coated Slide Hybridization

Hybridization probes derived from mRNA isolated from samples are employed for screening polynucleotides of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMASPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester N.Y.) is exposed to the membrane overnight at −70C, developed, and examined visually.

Polymer Coated Slide-Based Hybridization

Probe is heated to 65C for five min, centrifuged five min at 9400 rpm in a 5415C microcentrifuge (Eppendorf Scientific, Westbury N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C in 1×SSC, 0.1% SDS, and three times for 10 min each at 45C in 0.1×SSC in a dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of polynucleotides in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/ID) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively.

Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

X Complementary Molecules

Molecules complementary to the polynucleotide, from about 5 (PNA) to about 5000 bp (complement of an entire cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame.

Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the polynucleotide encoding the protein.

XI Protein Expression

SEQ ID NO: 1, the 577 amino acid protein encoded by SEQ ID NO: 17, is characterized by a potential AMP-binding domain from N82-V493 and transmembrane domains at V111-T137, M257-S276, and W265-F284. The expression profile for SEQ ID NO: 17 indicates that this molecule is differentially expressed in renal cell carcinoma.

SEQ ID NO: 2, the 552 amino acid protein encoded by SEQ ID NO: 18, is characterized by potential N-glycosylation site at N39, N56, and N102; transmembrane domains at F204-M222 and W357-M383; and transporter signatures at N102-K145 and R434-G483.

These proteins may be expressed by transforming the vector containing the cDNA into competent E. coli cells using protocols well known in the art (Ausubel, supra, unit 16, incorporated by reference).

Expression and purification of the protein are achieved using either a cell expression system or an insect cell expression system. The pUB6/V5-His vector system (Invitrogen, Carlsbad Calif.) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica califomica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

XII Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using a 431A peptide synthesizer (Applied Biosystems) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the Polynucleotide or Protein

The polynucleotide or the protein are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled polynucleotide or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories, Palo Alto Calif.), is used to screen for peptides that bind the protein of the invention. A polynucleotide encoding the protein is inserted into the multiple cloning site of a pLexA vector, ligated, and transformed into E. coli. A cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ]cells using a polyethylene glycol/lithium acetate protocol. Transformed yeast cells are plated on synthetic dropout (SD) media lacking histidine (-His), tryptophan (-Trp), and uracil (-Ura), and incubated at 30C until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30C. A sample of the culture is plated on SD/-Trp/-Ura media and incubated at 30C until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gall-Trp/-Ura, and white colonies are isolated and propagated. The pB42AD-cDNA plasmid, which contains a polynucleotide encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A combination comprising a plurality of polynucleotides wherein the plurality of polynucleotides have the nucleic acid sequences of SEQ ID NOs: 3-18 or the complements thereof.

2. An isolated polynucleotide comprising a nucleic acid sequence selected from SEQ ID NOs: 3-18 and the complements thereof.

3. A method of using a combination to screen a plurality of molecules to identify at least one ligand which specifically binds a polynucleotide of the combination, the method comprising:
a) contacting the combination of claim 1 with molecules under conditions to allow specific binding; and
b) detecting specific binding, thereby identifying a ligand which specifically binds the polynucleotide.

4. The method of claim 3 wherein the plurality of molecules or compounds are selected from DNA molecules, peptides, peptide nucleic acid molecules, repressors, RNA molecules, and transcription factors.

5. A method for using a combination to detect expression in a sample containing nucleic acids, the method comprising:
a) hybridizing the combination of claim 1 to the nucleic acids under conditions for formation of one or more hybridization complexes; and
b) detecting hybridization complex formation, wherein complex formation indicates expression in the sample.

6. The method of claim 5 wherein the polynucleotides of the combination are attached to a substrate.

7. The method of claim 5 wherein the sample is from kidney.

8. The method of claim 5 wherein the nucleic acids of the sample are amplified prior to hybridization.

9. The method of claim 5 wherein the comparison with standards assesses kidney function.

10. A composition comprising a polynucleotide of claim 2.

11. A vector comprising a polynucleotide of claim 2.

12. A host cell comprising the vector of claim 11.

13. A method for using a host cell to produce a protein, the method comprising:
a) culturing the host cell of claim 12 under conditions for expression of the protein; and
b) recovering the protein from cell culture.

14. A purified protein comprising a polypeptide having an amino acid sequence of SEQ ID NO: 1 or SEQ ID NO: 2.

15. A composition comprising the protein of claim 14.

16. A method for using a protein to screen a plurality of molecules to identify at least one ligand which specifically binds the protein, the method comprising:
a) combining the protein of claim 14 with the plurality of molecules under conditions to allow specific binding; and
b) detecting specific binding, thereby identifying a ligand which specifically binds the protein.

17. The method of claim 18 wherein the plurality of molecules is selected from agonists, antagonists, antibodies, DNA molecules, peptides, peptide nucleic acids, proteins, and RNA molecules.

18. A method of using a protein to screen a plurality of antibodies to identify an antibody which specifically binds the protein, the method comprising:
a) contacting a plurality of antibodies with the protein of claim 14 under conditions to form an antibody:protein complex, and
b) dissociating the antibody from the antibody:protein complex, thereby obtaining antibody which specifically binds the protein.

19. A method for preparing a polyclonal antibody, the method comprising:
a) immunizing a animal with protein of claim 14 under conditions to elicit an antibody response,
b) isolating animal antibodies,
c) attaching the protein to a substrate,
d) contacting the substrate with isolated antibodies under conditions to allow specific binding to the protein, and
e) dissociating the antibodies from the protein, thereby obtaining purified polyclonal antibodies.

20. An antibody which specifically binds a protein produced by the method of claim 18.


SEQUENCE LISTING (18 sequences)

SEQ ID NO: 1 - protein, 577 aa, Homo sapiens
  misc_feature: Incyte ID No 279978
MHWLRKVQGLCTLWGTQMSSRTLYINSRQLVSLQWGHQEVPAKFNFASDVLDHWADMEKAGKRLPSPALWWVNGKGKELMWNFRELSENSQQAANVLSGACGLQRGDRVAVMLPRVPEWWLVILGCIRAGLIFMPGTIQMKSTDILYRLQMSKAKAIVAGDEVIQEVDTVASECPSLRIKLLVSEKSCDGWLNFKKLLNEASTTHHCVETGSQEASAIYFTSGTSGLPKMAEHSYSSLGLKAKMDAGWTGLQASDIMWTISDTGWILNILGSLLESWTLGACTFVHLLPKFDPLVILKTLSSYPIKSMMGAPIVYRMLLQQDLSSYKFPHLQNCLAGGESLLPETLENWRAQTGLDIREFYGQTETGLTCMVSKTMKIKPGYMGTAASCYDVQVIDDKGNVLPPGTEGDIGIRVKPIRPIGIFSGYVENPDKTAANIRGDFWLLGDRGIKDEDGYFQFMGRADDIINSSGYRIGPSEVENALMKHPAVVETAVISSPDPVRGEVVKAFVILASQFLSHDPEQLTKELQQHVKSVTAPYKYPRKIEFVLNLPKTVTGKIQRTKLRDKEWKMSGKARAQ

SEQ ID NO: 2 - protein, 552 aa, Homo sapiens
  misc_feature: Incyte ID No 210710
MAFSELLDLVGGLGRFQVLQTMALMVSIMWLCTQSMLENFSAAVPSHRCWAPLLDNSTAQASILGSLSPEALLAISIPPGPNQRPHQCRRFRQPQWQLLDPNATATSWSEADTEPCVDGWVYDRSIFTSTIVAKWNLVCDSHALKPMAQSIYLAGILVGAAACGPASDRFGRRLVLTWSYLQMAVMGTAAAFAPAFPVYCLFRFLLAFAVAGVMMNTGTLLMEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQ

SEQ ID NO: 3 - DNA, 726 bp, Homo sapiens
  misc_feature: Incyte ID No 004516.1 Incyte Unique
tgaaaagattcattaaggtgcatgctttgatttaacatctgcaaacatttaaaaaatataacagtgtgtgacgtagcagtgagagtactatctttttttaaaaagggaaattaagatttatttctggccaatgtgaacagaaataagtcactttatctcactgagcaccaattttacacgtggaaaatagaagaattggaccaaatcagcagtttcaagctgagttgcaaaagttcatggaaccatttcagtcgcttcatggaatcggggtaggcatgaggcccgttgttctttcaacctgagccgcatggctcttgtgtctttaaaccttgtggtaggatttttttttttttctgctttaataagtgaagggtcagggcaccagttggtgtctgagatgccctccagtctggggaaccccgtagatgcttagactactttgaactgaagtatgtgcagtctgccatctcacattaaaatgtaggcattttgtcaattgcttttctttcatctgcacaagaggaaggagagaacgaatcaatacaaccactcttttccttgagactgcaaagaaaatggttctatagtttgatggttctacttcccagatgctacctctcagatttattctcaacagaaaattttttgattacagcagaccagatctttatctgtcaataagttaaaaaagataatctgggctggatgtggtggctcacgcctgtt

SEQ ID NO: 4 - DNA, 503 bp, Homo sapiens
  misc_feature: Incyte ID No 249553.1 Incyte Unique
agggagagaaaaaaattgtaaaaataaaaatagtaaaagaaactgataaagaaaagtaatggaagacaggaagaaaagaagagaaggaagtaaagaggaaaacttataaatattcccacagatagacaaagtcaagcataaaactggagcttgagaaggaaatgaaaggccgtggcaccttcttataccctagaagaagacctccatacaggaagacttgtgtgtggggttgggacattagaatcatccacaagtcaccccaaaccttggaactgtcagggtcagaggggaaccaccatttattaagcatttgccatgtgccaggcactaacccagatgcattataaataccacgttgtttcacctgtgtgtggcatctacagaccttagatcatagctgtgagaacaacgtaagcactgccaaagttatcagctacccatatctcatgtttttgatgttatctactcttcctagaatcaaatattaaaataattttaaaacc

SEQ ID NO: 5 - DNA, 543 bp, Homo sapiens
  misc_feature: Incyte ID No 213764.1 Incyte Unique
agcctccatttttctccagatggttgaaataaccagcctctgaaggagcccaatggtttggtcactgctctctcagcaaattacagtcactgtcacttagcatggagagtggacgttgcacatcactgtgaaaccttgcagaggaggagagggcaggttcatcagaagaaagaaaggacaaaatgactccttatgaagcattttgtgccttctgtgagaaaacatgtatttagatcagataaactctagtcaaaatacaaaaaggaaaaatgaaagacctctgaaataggaacaatctcttgaagaggcaaatgactcaaaactgctcagtggctctttcagaaaatctaagtaaagttccctgacaacagaaactgaagagattgcctggttcatcttgtagtcttccaaaacagcagataatttctgaatctcagatgttgaatcagtgcaacgggatggatttcttgttcctaagtgttaaatgatcacatacataaaagagtctgagcctgagcaacatagtagagaccctgtctctac

SEQ ID NO: 6 - DNA, 1245 bp, Homo sapiens
  misc_feature: Incyte ID No 108833.1 Incyte Unique
tgaatggggccttttttaggtcctagttaccaatacttcccatctcccaaaattctctgatagctcgtatgtttattcatttattcatctattgnnnnnnnnnnnnnnnnnnnnnnnnnnnnnnnnnnnnnnnatgtgtggtcgtttcattgtatggtgatgagccaggcagatatggtttctggttttaacaaagagatgtttaacaagagtataacaggtagacctggtatctgtggagtcagggaaagtttccatgaggaagggacatttaaactgagataagaagtagctggatgaagtggggaggagggcatgtaggtgtgaggaccatgggtgctggaattttgaaaatggtaatcaaccacagcatgtgcaaaagtcctgcagtgggaaggaacacgatggactctaggtgaatgagagattcagagagtaactggagatggttctggagacatgggcagggccaaatcacacagggttttgtaagccatgtcagataagaattttaaactctatcttaagatcaatgggaagccactgataaggcaggagagtggcataagcaggttcatatttttaaatgtcactctgtctacactgtggagaatgggtgggaggggaaagtaaaaggatgaaggaagaccaattataaggacacaacaaaagcacaagtgagagatgatgttgagaccaggatagtggggtcacgatcaggatggagagaagcagccagatatgagatctttaggaaacagaatgatcagaattatcaggtgataggttagatgtgagtgcatggagagggtaaggaggccaggtagattcctgggtttctcttgaacaattaggcagacaatggggtaaagttagtaccgtttgctgaattagggtagccaggagcagaagcaggtttgtgagagaaagagaaggtagggctgcattattgccaagtagatatcaaagtggaggtgtccagttttacaaaaacgtcctttgtagcttaggaaagagatcagggctgcagacgttaagatggttatctgccagtgctatctaaataggcaacaacaatcattataatagtattcaaggggaaaaactggtagcctctcactgaggcccaagaaatcacaaggcatgaattctcacattaaaatgcttgtttccaactcaccaggctgtatacattcaatatgtgtagcttttgtatgtcaatcttagttcaataaagtagttttt

SEQ ID NO: 7 - DNA, 656 bp, Homo sapiens
  misc_feature: Incyte ID No 004742.1 Incyte Unique
gacagggtaaaggaaattggaaaaacccataagatgtatttgaggttctttctattccagggaattcttggtatgataggacatgaaagctgagagaaaacccttctacctaggaactccggagtttcaactaccagagactgaaggcaggagagaaaaactaagacaaatgacttcaaagcctttctgaaatgccacagagggtcaggattacaatctgcctattctgattcttctactatgggaaaaaatctttttcagaattgatgtaaagcctctgttaatacttttcctgttaacaacctaactattcaactgctgaataaaaaccattaggacaactaaagaaactgagagattttggcaagaaatagtcattccaatatcaatgactacggagggaatgcaattacttttctttgcttaagttttccaggtttgtgttcttaagtatgagccacagcatttataacaaccccagaatgtaccatttttatacaagaattaaataatagcccaaattaagtatttggctcttaggaatttgagaacttttgcaaaatgatatctttcataaaaaataaatgttgtaaaactatatatatttaataaacccattgcagtaaccagcaaaaataaatttagctttatgaaaa

SEQ ID NO: 8 - DNA, 484 bp, Homo sapiens
  misc_feature: Incyte ID No 980289.2 Incyte Unique
ctttgtccatgcttctgcagggtgtaaaagtaaaaatcctatacttcccatattcaacgtttagattttaaacaactgaacaagcactttcacaattagccttcctcagattggaatgcgaagtcaagccaacgtgtacctcttactgcagaagatttctgcactgtgaatgtgataggatttgcctccttaaccagagggtgctggtttttcctggctggagggtagaaggtcatgaatagaggacaaagcacagggaaggggcagcatgtggggaagagccccaaggatctcagtatgaaaataaagaggtgtgagccaactgcccataggtttgggctatgagtctggggaaactgtttcaaaatcaatggaggcccaaaggcagagggaaaattctgtgtatggacttccatgtcattcagaaatgttaactccttgaaaagagttaatatatttttttcttttttaaacatttcaatag

SEQ ID NO: 9 - DNA, 615 bp, Homo sapiens
  misc_feature: Incyte ID No 980289.1 Incyte Unique
ctcaggaaccttatagccagaatgagggagaggtgagtactaactccacagtgttatcccagtcgctactcatttcccttaccaccaccccttcaaacacttcagtgtactttcagctctcaagaaaaatgcaaactttcatctgggcctaagaatctctatatgaactacccctgctcacctccggagaggcatccccttccaccaccctcccctccttcttctctatcctcattgctcctttcaatggtcctgacaggatccctttcagccacaggcccttagcaaatgctgctctcacggcccaagtcatcttccctggttcatgcctacttcggattaaagttacccctccggagagtgttcccaatcaatctgagttgaccaagtccttattacacactctcaaactccatgagatgtccttcatagctctcatcccagttataattagtttgcatttatttacacaatggctgtattaatgtctgtattctccaccagaatgagagcttcatgagagccaggatgtgtgttttctcaaaatctgctttattgtggtctaatttgtgtataataaacacacccatgttaggtgtacccatcaatgaaaaa

SEQ ID NO: 10 - DNA, 1342 bp, Homo sapiens
  misc_feature: Incyte ID No 071972.1 Incyte Unique
ttgagaatggtgcttcaatttaattgatttcctttataatctgtgtaaatattttattttaaggcatttaaaagtattatttctgaggagggatgtacaagcttcaccagagagctgagtagggtttataaattcaaaaaatggtaagaaacttgatggaaaggcttgcccagggcagcaggcagcccagcacctgaggggaggagtaagacatgggcatggttagccaagggccaattgaaactaggacacaagtgtgactaccttttatctcaagaggcaactagatgactatgaaccttaccatgagacggtgattcactttctcgttccataagttgaaaagatattttggaagcttgctgtgtgtcgggctctgtggagtttcacagtaaatagaaggaacaaaaacctctgcccctncacacagcttatattctaaagggggagggccaactattaaatgaaagtgaaatatatagtatgttaatgacaaagaggttgtagaagaaacaaagcaggagaagaaagacatttgcaattttaattgcattgtcagaaaaagcattcctgagagacagtggtctgtttctgcctggttacaccaagggaatattttcaggtgagtgccaatgtgatgttcacacgtctttggaatctccctttgcaataaaaataaagcagaagtctgtttcagagcattttgcaattagcagagtctaactcaaacttaatacaactcttttgttgatcttgtcctcattgtatggtcctgttcgtatgttatgcaagtgatacttgtctgccatttgaattcatgacctaaagattcttctagtaagtaaatgtatttcagtaaaaaatacaagtggatttaaagaaaaatagtaagtaaataataatacagatgttgggacacacaggaaaacgctcataaaggtggtatctcaataaagaaaaaaagaactgaaagttggaaactgttggcatgaccttgattggagtatcacagagatttgggttcaaatcttggctttgccatttcttagctttaagaaacaggttagctcatctctctgggttttttctattctcagacgtaaatggggataaattaatacctgctatgctgggattttaaaacttgtttttcatgaggattacaggtgtgagccaccacgcctggctgaaaaattgcatctttatattcagttacctcttctaatgctgaaaccttagtcattcccatccattataaatacaggtcaacaagactacagaaatattagcaggttgtgtgaacgccatttatacctcatcagctactttttagttactataa

SEQ ID NO: 11 - DNA, 933 bp, Homo sapiens
  misc_feature: Incyte ID No 071870.1 Incyte Unique
aaaataaaataaaagtacagcagggacattgcggaaaacttggaaagttttgaaaaagaagcaggaaaaaatgtagggtgtgtctataagttgccatctgggtgagtgtgggagctggggtgagaactaccagtgtaggataggtgtcagaacatcacaaggcatgaaaagaggagacaagtcggggagacagtgaaagtgactggaggggacacatggatgtccaggttggcttgagcacgtggagccctcacaggccatactgtctgcctttcaggagttgagtccacttaacctcagggcttttctcttgccaggcagagtctgcacttttggagccagatctgaattatgggagacacagagctaagagtgaaaaacactcccagaagctcccgtctcagttttgcagtcagtgttcagcctccctccgaaatcaactttgaggaaagagtgcggaatggcagagggggtgcccagtttgcctccccagaaagcctatgggtatcctgacccagcccagcacatgtgggagtctctgcatgctctatttcgggtttctccttctaactgtgtttgggtgcaattgtgtatacgtgcagatgggtgcacacactccaatttcatcagtggctctcggtacccagaggtttccattgttataattataccaggcatattgtagatagcacatagcagctaataatttttgaatgtcattgctgggaaatcaggaagtgctgacttttggatagtttcagctctgcactgatgacagtttcactttagtatcaaatatataaagcacctatggcatgctacacacagttctacgcactttgagaaattaacgaatttaatccttacagctatcttatcacatgggctgcgatcatttcttatgtacagatggagaaattgaggtacacagag

SEQ ID NO: 12 - DNA, 1045 bp, Homo sapiens
  misc_feature: Incyte ID No 311180.1 Incyte Unique
ttgggagcagagctgatttccaatcttcctgctgccctcgctgcctgtgtgtgccgtggccttcttgagctttagttttctcacctgtggaacaggtttcatgagcagttggaaggaagcaaatgagcttcattttctacaacagggtgggtggtgtagttgatattcaccccaaaggtgcaggatccccacttctgtttattgccagcgctccgagtttgcttctgcatggaaggggaagatggactagagctgatacctcctgcaccctggtgaagaaatgcaccctgaggccagaaaatagtacaaacccatctggctgcaacagtccagcagcatgaagatcaaagtcacgtagtagagccagagagaaatttgccctgggcctaatattcatgaagcctttccaattcagactcggtattattgccaagtcggtgtgtttgggagaaatcagaagccaacacactagaaaagttgcaagagattcctaggggaacccacaaattcagttccttgccagaaacctgctcctccagatgaatctagtgcatcagatgggctcagaagcacaggtggcgcaggcagacctctactgagaagaacaacagtggaaggcagatgaaactcacattttggagtctgagagccatgcgggcccaccccagttacgtctacccgtggtcggtgaccagagaatcctttagcagcaacagcagtgatgtttatttcactggtatgtataaagcgactgattgaaaacaagctgtatgcccaacaacttggaaatggttaaatgatttttggttcagccgcacagtggaatatcatgcggccattaagaataaataaaaaatcctatactttccatattcaactttagactgcaaacaactgaaaaaacaccttcataattaggcttcctcggattagaacgtgaagtcaaatcaacgtgtgtcagaaagttaacaacagctaactgtttggttggtggtgttctgagtaacttgtattattgtgcagttttttgcccattcgtaatgtg

SEQ ID NO: 13 - DNA, 511 bp, Homo sapiens
  misc_feature: Incyte ID No 393706.2 Incyte Unique
tggtcagccaggagctgtgggcagggctacctgggaagagggccctaaagggtccatccagagcccccacggggccccggtgggggtctcatgcagcatctgacccgccctctcctctcctggggcatcccctcggcagcctcagcctggcctctatctgcactggtgttgctaggtgactctgagggggttcccagagtgtctcatccttcgtgtgggcaggtctcaggagtggccagcagcaaaccccgtaccgcagtcttcgccagatgcccttggcgtactgtaggaggtttgctttctctgggagccctttagagtccggagggacttggccttggcctgcccttaaggctgagtttagagctttccactcatactcttccttcctctcccacatttcttgatctccaccccacccccatgccagccacccccatgccagccacctccctggaaaccagggatacagaaataaacaagacctggccctggtctgccaggggctcgc

SEQ ID NO: 14 - DNA, 623 bp, Homo sapiens
  misc_feature: Incyte ID No 405479.1 Incyte Unique
aatttaatgaactctaacaggatttttgtttctaaacccaggacattgagatctgctggttggagatcttagtgcccaaggaagaatgcttgcaaaaggagatggaacagtgtttctgttgcattggccactgagaatatctggttattttgaatttgtcacagtctgcagctaaaaggcgaacaggaaaagaagaggaagcggcacatgagactagcaggagtaaatgttatatacactgggcctcgcaaaaattctgcaatcactgggaatcagtaacctggtagaatgagaaaacaacgtaatactgaaaaatcaaaacaccatcctacagaactttgatgcattcagaacaaagatcacgaatcaagctgaaaaagtaaagcattctgtgagttgtggaatggaaattgcctcaagcatctcaccacttgatgacacttgtaatttcagtgacccaattcaaggaattcactgaatgtctatctatcatgtatgctgttggaggcacctgtctttcagttcagaggttatggatgtatctatcatcagtatatcgggaagtcagggacccggaacagagggacttaatgaagctgttggcgaagaaaaaattatgaaga

SEQ ID NO: 15 - DNA, 519 bp, Homo sapiens
  misc_feature: Incyte ID No 413721.1 Incyte Unique
agagggagaaagggagagaaaaaaattgtaataataaaaatagtaaaagaaactgataaagaaaagtcatggaagacaggaagaaaggaagagagggaagtaaagaggaaaacctataaatattcccacagatagacaaagtcaagcataaaactggagcttgagaaggaaatgaaaggaaaagaagtggtactttcttttaccctaggagaagacctccatacaggaagacttgtgtgtggggctgggacattagaatcatccgcaagtcactccaaaccttgaaactgtcagggtcagaggggaactaccatttattaagcatttgccatgtgccaggcactgacccagacacattataaataccacattgtttcacctgtgtgtggcatctacagaccttagatcatagctgtgagaacaaagtaagcactgccaaagttatcagctacccatacctcatgtttttggtgttatctactcttcctagaatcaaatattaaaataattttaaaacca

SEQ ID NO: 16 - DNA, 840 bp, Homo sapiens
  misc_feature: Incyte ID No 334440.1 Incyte Unique
caggcataagccaccgcgcccagccagaggcaacattttttaacgcagttatcattctaggaaatttataggtcctttgaaggaaaattctgtgggcaaataagattgtgatacatggtatttcagttttcccaaatgtggccagcccgatctggtcaaaaattttattttttaaaagctatagtgtctttttttcttaaatttgaggcaacatgcacaaaattggagatttgaaattaaagccaagatttgtagtttctctggaaagacctggcaagattggactggattgctatgtgaccagggtcccactagatggggctgcatcctctaatccccaaatccttatgttccctgcatgctcacctttgttacctgcctgacacctgtggggcttttaactttatggcaactgccctattctctggatccttcctgaggatttatgatgcgtaatactccaggaatctggttagctttgcttaacacatttccaaaacttgtttgaatgcatgagtacagtcactagtagcattctgtgcagtacaatgtatgggggcttaggagtttagggtagtatacaggattagggataggacttgagtctaatcctaactcttagcagttacactggatgacattagagcaaatggttctttacgtctacattttcttcatctgtagatgtaataatttccatatcaactatgatgtacagtgctaattccaatgaaatgttacatgtgagaagtctttgaaatgtaaaaaacactacagatactgaagcagtttggagaattaaaaaacactacgaaaacacagcttggtatctgtagtgtt

SEQ ID NO: 17 - DNA, 2046 bp, Homo sapiens
  misc_feature: Incyte ID No 279978
gtgctctcttccaaggctgtaggagttctggagctgctggctggagaggagggtggacgaagctctctccagaaagacatcctgagaggacttggcaggcctgaacatgcattggctgcgaaaagttcagggactttgcaccctgtggggtactcagatgtccagccgcactctctacattaatagtaggcaactggtgtccctgcagtggggccaccaggaagtgccggccaagtttaactttgctagtgatgtgttggatcactgggctgacatggagaaggctggcaagcgactcccaagcccagccctgtggtgggtgaatgggaaggggaaggaattaatgtggaatttcagagaactgagtgaaaacagccagcaggcagccaacgtcctctcgggagcctgtggcctgcagcgtggggatcgtgtggcagtgatgctgccccgagtgcctgagtggtggctggtgatcctgggctgcattcgagcaggtctcatctttatgcctggaaccatccagatgaaatccactgacatactgtataggttgcagatgtctaaggccaaggctattgttgctggggatgaagtcatccaagaagtggacacagtggcatctgaatgtccttctctgagaattaagctactggtgtctgagaaaagctgcgatgggtggctgaacttcaagaaactactaaatgaggcatccaccactcatcactgtgtggagactggaagccaggaagcatctgccatctacttcactagtgggaccagtggtcttcccaagatggcagaacattcctactcgagcctgggcctcaaggccaagatggatgctggttggacaggcctgcaagcctctgatataatgtggaccatatcagacacaggttggatactgaacatcttgggctcacttttggaatcttggacattaggagcatgcacatttgttcatctcttgccaaagtttgacccactggttattctaaagacactctccagttatccaatcaagagtatgatgggtgcccctattgtttaccggatgttgctacagcaggatctttccagttacaagttcccccatctacagaactgcctcgctggaggggagtcccttcttccagaaactctggagaactggagggcccagacaggactggacatccgagaattctatggccagacagaaacgggattaacttgcatggtttccaagacaatgaaaatcaaaccaggatacatgggaacggctgcttcctgttatgatgtacaggttatagatgataagggcaacgtcctgccccccggcacagaaggagacattggcatcagggtcaaacccatcaggcctataggcatcttctctggctatgtggaaaatcccgacaagacagcagccaacattcgaggagacttttggctccttggagaccggggaatcaaagatgaagatgggtatttccagtttatgggacgggcagatgatatcattaactccagcgggtaccggattggaccctcggaggtagagaatgcactgatgaagcaccctgctgtggttgagacggctgtgatcagcagcccagaccccgtccgaggagaggtggtgaaggcatttgtgatactggcctcgcagttcctatcccatgacccagaacagctcaccaaggagctgcagcagcatgtgaagtcagtgacagccccatacaagtacccaagaaagatagagtttgtcttgaacctgcccaagactgtcacagggaaaattcaacgaaccaaacttcgagacaaggagtggaagatgtccggaaaagcccgtgcgcagtgaggcgtctaggagacattcatttggattcccctcttctttctctttcttttccctttgggcccttggccttactatgatgatatgagattctttatgaaagaacatgaatgtaagttttgtcttgccctggttattagcacaaaacattactatgttagatattgaaataaggaagaaaagaaagaggagatgaaagggggagaaaa

SEQ ID NO: 18 - DNA, 1680 bp, Homo sapiens
  misc_feature: Incyte ID No 210710
catggcattttctgaactcctggacctcgtgggtggcctgggcaggttccaggttctccagacgatggctctgatggtctccatcatgtggctgtgtacccagagcatgctggagaacttctcggccgccgtgcccagccaccgctgctgggcacccctcctggacaacagcacggctcaggccagcatcctagggagcttgagtcctgaggccctcctggctatttccatcccgccgggccccaaccagaggccccaccagtgccgccgcttccgccagccacagtggcagctcttggaccccaatgccacggccaccagctggagcgaggccgacacggagccgtgtgtggatggctgggtctatgaccgcagcatcttcacctccacaatcgtggccaagtggaacctcgtgtgtgactctcatgctctgaagcccatggcccagtccatctacctggctgggattctggtgggagctgctgcgtgcggccctgcctcagacaggtttgggcgcaggctggtgctaacctggagctaccttcagatggctgtgatgggtacggcagctgccttcgcccctgccttccccgtgtactgcctgttccgcttcctgttggcctttgccgtggcaggcgtcatgatgaacacgggcactctcctgatggagtggacggcggcacgggcccgacccttggtgatgaccttgaactctctgggcttcagcttcggccatggcctgacagctgcagtggcctacggtgtgcgggactggacactgctgcagctggtggtctcggtccccttcttcctctgctttttgtactcctggtggctggcagagtcggcacgatggctcctcaccacaggcaggctggattggggcctgcaggagctgtggagggtggctgccatcaacggaaagggggcagtgcaggacaccctgacccctgaggtcttgctttcagccatgcgggaggagctgagcatgggccagcctcctgccagcctgggcaccctgctccgcatgcccggactgcgcttccggacctgtatctccacgttgtgctggttcgcctttggcttcaccttcttcggcctggccctggacctgcaggccctgggcagcaacatcttcctgctccaaatgttcattggtgtcgtggacatcccagccaagatgggcgccctgctgctgctgagccacctgggccgccgccccacgctggccgcatccctgttgctggcggggctctgcattctggccaacacgctggtgccccacgaaatgggggctctgcgctcagccttggccgtgctggggctgggcggggtgggggctgccttcacctgcatcaccatctacagcagcgagctcttccccactgtgctcaggatgacggcagtgggcttgggccagatggcagcccgtggaggagccatcctggggcctctggtccggctgctgggtgtccatggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttttagcctcctggggaacctgc